(19) United States
(12) Patent Application Publication
(10) Pub. No.: US 2025/0223626 A1
(43) Pub. Date: Jul. 10, 2025

(54) METHODS OF DIAGNOSING IBS-D AND SELECTION OF IBS-D TREATMENT

(71) Applicant: Cedars-Sinai Medical Center, Los Angeles, CA (US)

(72) Inventors: Mark Pimentel, Los Angeles, CA (US); Gabriela Leite, Porter Ranch, CA (US); Ruchi Mathur, Los Angeles, CA (US); Ali Rezaie, Beverly Hills, CA (US); Walter Morales, Rowland Heights, CA (US); Stacy Weitsman, Los Angeles, CA (US)

(73) Assignee: Cedars-Sinai Medical Center, Los Angeles, CA (US)

(21) Appl. No.: 18/852,803

(22) PCT Filed: Apr. 18, 2023
(86) PCT No.: PCT/US2023/065904

§ 371 Date: Sep. 30, 2024

Related U.S. Application Data
(60) Provisional application No. 63/333,007, filed on Apr. 20, 2022.

(51) Int. Cl. C12Q 1/06; A61K 31/197; A61K 31/395; G01N 33/497

(52) U.S. Cl. CPC C12Q 1/06; A61K 31/197; A61K 31/395; G01N 33/4975; G01N 2800/065; G01N 2800/52

(57) ABSTRACT

The present invention describes methods of detecting IBS-D. Further described are methods selecting a therapy and methods of treatment for IBS-D. These methods are based, at least in part, on a subject's level of Desulfovibrio, Fusobacterium, or hydrogen sulfide.

### CROSS-REFERENCE TO RELATED APPLICATIONS

This application includes a claim of priority under to U.S. provisional patent application No. 63/333,007, filed Apr. 20, 2022, the entirety of which is hereby incorporated by reference.

### FIELD OF INVENTION

This invention relates to treatment and detection of irritable bowel syndrome.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Irritable bowel syndrome (IBS) is estimated to affect one in ten people globally, with a prevalence of 11.8-14.0% in North America. IBS subjects suffer from abdominal pain, bloating, and have alterations in stool form and frequency that occur for at least 6 months. Based on the predominant stool pattern, IBS is divided into three main subtypes, constipation-predominant (IBS-C), diarrhea-predominant (IBS-D), and mixed constipation and diarrhea (IBS-M).

While the etiology of IBS remains incompletely understood, there is a growing body of literature suggesting a role for the intestinal microbiome. However, specific findings have been inconsistent between studies. This may be due to the heterogeneous nature of IBS, and grouping different IBS subtypes together. Despite this, one consistent finding is the association between small intestinal bacterial overgrowth (SIBO) and the IBS-D subtype, based on a recent large meta-analysis of both breath testing and small bowel culture studies. A diagnosis of SIBO is based on the presence of increased hydrogen (H₂) on breath test, and studies have found that SIBO is present in over 80% of IBS-D subjects. However, new data suggest that another gas produced by gut microbes, hydrogen sulfide (H₂S), is associated with a diarrhea phenotype.

The microbiome is important to the understanding of IBS, and breath testing (hydrogen, methane and now hydrogen sulfide) has an important role. Discussed herein, we examine the role of archaea and sulfate-reducing bacteria in the mechanisms of diarrhea and constipation in IBS, and describe therapies and diagnostics based on these roles, among other things.

### SUMMARY OF THE INVENTION

The following embodiments and aspects thereof are described and illustrated in conjunction with compositions and methods which are meant to be exemplary and illustrative, not limiting in scope.

Various embodiments provide for a method of distinguishing IBS-D from IBS-C in a subject, comprising: detecting a quantity of Desulfovibrio, a quantity of Fusobacterium, or both in a biological sample from the subject; and identifying the subject as having IBS-D when the quantity of Desulfovibrio, the quantity of Fusobacterium, or both are each higher than its reference quantity.

In various embodiments, the method can further comprise administering an IBS-D therapy. In various embodiments, the IBS-D therapy can comprise rifaximin. In various embodiments, the IBS-D therapy can further comprise N-acetyl cysteine (NAC).

Various embodiments of the invention provide for a method of selecting an IBS therapy for a subject, comprising: detecting a quantity of Desulfovibrio, a quantity of Fusobacterium, or both in a biological sample from the subject; and selecting an IBS-D therapy for the subject when the quantity of Desulfovibrio, the quantity of Fusobacterium, or both are each higher than its reference quantity.

In various embodiments, the IBS-D therapy can comprise rifaximin. In various embodiments, the IBS-D therapy can further comprise N-acetyl cysteine (NAC).

In various embodiments, the biological sample can be a stool sample. In various embodiments, the method can further comprise administering the IBS-D therapy.

Various embodiments provide for a method of predicting a subject's response to an IBS-D therapy comprising rifaximin, comprising: detecting a quantity of Desulfovibrio, a quantity of Fusobacterium, or both in a biological sample from the subject; and characterizing the subject as a responder to the IBS-D therapy when the quantity of Desulfovibrio, the quantity of Fusobacterium, or both are each higher than its reference quantity.

In various embodiments, the IBS-D therapy can further comprise N-acetyl cysteine (NAC). In various embodiments, can further comprise administering the IBS therapy.

In various embodiments, the biological sample can be a stool sample.

Various embodiments of the invention provide for a method of predicting a subject's response to an IBS-D therapy comprising rifaximin, comprising: detecting a quantity of hydrogen sulfide (H₂S) in a biological sample from the subject; and characterizing the subject as a responder to the IBS-D therapy when the quantity of H₂S is higher than its reference quantity.

In various embodiments, the IBS-D therapy can further comprise N-acetyl cysteine (NAC).

In various embodiments, the biological sample is a breath sample. In various embodiments, the biological sample is whole blood, serum, or plasma.

Various embodiments of the invention provide for a method of treating IBS-D, comprising: administering an IBS-D therapy comprising rifaximin or an IBS-D therapy comprising rifaximin and N-acetyl cysteine (NAC) to a subject who has been identified as a responder to the IBS-D therapy by a method selected from: (i) detecting a quantity of Desulfovibrio, a quantity of Fusobacterium, or both in a biological sample from the subject, and characterizing the subject as a responder to the IBS therapy when the quantity of Desulfovibrio, the quantity of Fusobacterium, or both are each higher than its reference quantity; or (ii) detecting a quantity of hydrogen sulfide in a biological sample from the subject, and characterizing the subject as a responder to the IBS-D therapy when the quantity of hydrogen sulfide is higher than its reference quantity.

In various embodiments, the IBS-D therapy can decrease hydrogen sulfide produced in the subject's gastrointestinal system. In various embodiments, IBS-D therapy can decrease the quantity of Desulfovibrio, a quantity of Fusobacterium, or both in the subject's gastrointestinal system.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1C shows breath test results in IBS-D and IBS-C subjects. (1A) Area under the curve (AUC) for breath H₂. (1B) Rates of positivity for SIBO based on H₂. (1C) AUC for H₂S. (D) AUC for CH₄. Data are shown as mean±SD. Statistical analyses by Mann Whitney U test.

FIGS. 2A-2B show associations between breath H₂, H2_{S} and CH₄ in pooled IBS-D and IBS-C/CH₄+ subjects. (2A) Association between H₂ and H₂S AUCs. (2B) Association between H₂ and CH₄ AUCs.

FIGS. 3A-3D show stool microbial alpha diversity indices in IBS-D and IBS-C/CH₄+ subjects (3A) Chao 1, (3B) Simpson's index, and (3C) Shannon's index. (3D) Associations between microbial alpha diversity indices and breath H₂, H₂S and CH₄ (Spearman R). Blue gradient (top)—positive correlations; red gradient (bottom)—negative correlations. Colors indicate ranges of correlation coefficients; circle sizes denote coefficients within each range (Spearman R). ***P<0.001, **P<0.01, *P<0.05.

FIGS. 4A-4C show the following: (4A) Top 25 bacterial families correlated with relative abundance of archaeal family Methanobacteriaceae (Spearman's test). (4B) Associations between microbial alpha diversity (Shannon's and Simpson's indices), breath H₂, CH₄ and H₂S AUC and bacterial families associated with Methanobacteriaceae. Blue gradient (top)—positive correlations; red gradient (bottom)—negative correlations. Colors indicate ranges of correlation coefficients; circle sizes denote correlation coefficients within each range (Spearman R). ***P<0.001, **P<0.01, *P<0.05. (4C) Top 25 bacterial families correlated with SRB family Fusobacteriaceae (Spearman's test).

FIGS. 5A-5D show changes in absolute levels of M. smithii (5A) and stool wet weights (5B) of rats on HFD for 7 weeks. (5C) Small bowel levels of M. smithii after lovastatin hydroxyacid and lovastatin lactone treatment. (5D) Changes in stool wet weight after 10 days of treatment with water (controls), lovastatin hydroxyacid or lovastatin lactone. Horizontal bars denote mean±SD.

FIGS. 6A-6C show change in stool wet weight in rats gavaged with (6A) D. piger and (6B) F. varium. P-values denote comparisons between SRB-gavaged rats and controls. (6C) H2S gas production in stool collected from D. piger- or F. varium-gavaged rats and controls. Horizontal bars denote mean±SD. Statistical analyses by Mann Whitney U test.

FIGS. 7A-B show animal Study Design. (7A) Investigation of the effects of diet-induced increases in the methanogen Methanobrevibacter smithii, and of methanogenesis inhibitors, on stool consistency. (7B) Investigation of the effects of gavage with H₂S producers Desulfovibrio piger and Fusobacterium varium on stool consistency.

FIGS. 8A-8D show breath H₂, H₂S and CH₄ levels during the breath test in all subjects. (8A) IBS-D, (8B) IBS-C, (8C) IBS-C/CH₄−, and (8D) IBS-C/CH₄+ subjects throughout the breath test. All data represent median values.

FIGS. 9A-9D show breath H₂, H₂S and CH₄ levels in IBS-D, IBS-C/CH₄− and IBS-C/CH₄+ subjects throughout the breath test. (9A) H₂ levels in all groups. (9B) Scatter plot of delta H₂ levels (comparing levels at 0 and 120 minutes). (9C) H₂S levels in all groups. (9D) CH₄ levels in all groups. *P<0.05; **P<0.01; ***P<0.001 IBS-D vs IBS-C/CH₄−; †P<0.05; ††P<0.01; †††P<0.001 IBS-D vs IBS-C/CH₄+; ‡P<0.05; ‡‡P<0.01; ‡‡‡P<0.001 IBS-C/CH₄− vs IBS-C/CH₄+.

FIG. 10 shows nonmetric multidimensional scaling (NMDS) ordination of fecal microbiota of IBS-D and IBS-C/CH₄+ subjects. The distance between OTUs was calculated with the Bray-Curtis index. Each circle represents a sample: red—IBS-C/CH₄+, blue—IBS-D. PERMANOVA P-value<0.001.

FIG. 11 shows relative abundances of bacterial phyla in stool samples from IBS-D and IBS/CH₄+ subjects.

FIG. 12 shows KEGG modules enriched in the stool of subjects with IBS-C/CH₄+ (red) and IBS-D subjects (green).

FIG. 13 shows a summary diagram showing the microbial subtypes in TBS.

FIG. 14 (panels A-F) show the following: Panels A and B—differences in stool wet weight (A) and serum CdtB antibodies (B) between CdtB-inoculated rats (N=55, grey) and controls (N=24, orange). Panels C to F represent ileal microbiome differences between CdtB-inoculated rats (N=55, green, blue and purple) and controls (N=24, orange): C—Ileal microbial beta-diversity and clustering analysis, D—ileal Escherichia coli absolute abundance corrected for bacterial total load, E—ileal microbial alpha-diversity represented by Shannon index and, F—ileal Desulfovibrio log transformed count in CdtB-inoculated rats and controls.

FIG. 15 shows ileal and stool microbiome profiles across controls and CdtB-inoculated rats from the 3 different clusters observed after beta-diversity clustering analysis. Each color represents the relative abundance of microbial families across groups. The ileal microbiome of CdtB-inoculated rats from cluster 3 harbors microbial taxa commonly found in stool—a classic signature of fecalization of the small bowel.

FIG. 16 shows a comparison of the area under the curve of hydrogen, hydrogen sulfide and methane between IBS-D and IBS-C subjects. Statistical analysis was performed using the Mann Whitney U test.

FIG. 17 (panels A-E) shows the following: A—Stool microbiome profile at genus level of IBS-D and IBS-C/CH4+ subjects. B—Nonmetric multidimensional scaling (NMDS) ordination of fecal microbiota of IBS-D and IBS-C/CH₄+ subjects. The distance between OTUs was calculated using the Bray-Curtis index. Ellipses represent each group; pink ellipse represents the IBS-C(CH₄+) group, and the blue ellipse the IBS-D cohort. Each circle represents a sample: pink circles represent the IBS-C/CH₄+ participants and blue circles represent the IBS-D subjects. The PERMANOVA test indicated statistical differences between groups P<0.001. C—Shannon diversity index in IBS-C(CH₄+) compared to IBS-D subjects. Comparison was performed with the Mann Whitney U test (P=0.023). D—LDA scores of microbial metabolic pathways enriched in IBS-C/CH₄+, including those associated with methanogenesis. E—LDA scores of microbial metabolic pathways enriched in IBS-D including associated with H₂S production.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein “substantial” when used in connection with a referenced numeric indication means an amount of at least 60% of the referenced numeric indication, unless otherwise specifically provided for herein. In various embodiments, the term “substantial” when used in connection with a referenced numeric indication can mean an amount of at least 65%, 70%, 75%, 80%, 85%, 90%, 95%, 96%, 97%, 98%, or 99% of the referenced numeric indication, if specifically provided for in the claims.

As used herein, the terms “treat,” “treatment,” “treating,” or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented. Non-limiting examples of treatments or therapeutic treatments include at least one selected from pharmacological therapies, biological therapies, interventional surgical treatments, and combinations thereof.

Examples of biological samples include but are not limited to body fluids, whole blood, plasma, serum, stool, intestinal fluids or aspirate including duodenal fluids or aspirate, stomach fluids or aspirate, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In particular embodiments of the method, the biological sample may be whole blood, blood plasma, blood serum, stool, intestinal fluid or aspirate including duodenum fluids or aspirate, stomach fluid, or aspirate, saliva. In various embodiments, the biological sample may be whole blood. In various embodiments, the biological sample may be serum. In various embodiments, the biological sample may be plasma. In various embodiments, the biological sample may be stool.

Although SIBO has been described in IBS-D, it is not associated with IBS-C. Data suggest that the gut microbiome in IBS-C is different from IBS-D, as IBS-C is associated with increased methanogenesis and intestinal methanogen colonization, now known as intestinal methanogen overgrowth (IMO). Interestingly, methane (CH₄) is directly linked to slowing of intestinal transit in an animal model and an increased motility index in methane-producing IBS subjects and may cause constipation.

These findings demonstrate that changes in the gut microbiome are not uniform in IBS as a whole. Rather, different microbial compositions may account for the differing phenotypes of IBS. Identifying these microbiome-based subtypes may more clearly define possible microbial pathomechanisms in IBS in general. In this study, we combine 3-gas (H₂, CH₄ and H₂S) breath testing, stool microbiome sequencing, and translational animal models to identify potential microbial drivers of clinical phenotypes in IBS subtypes.

Acute gastroenteritis causes irritable bowel syndrome (IBS) in up to 11% of affected individuals. In an animal model of post-infectious IBS, rats given Campylobacter jejuni develop altered stool form and small intestinal bacterial overgrowth (SIBO), dependent on the presence of cytolethal distending toxin B (CdtB), commonly produced by IBS-causing microorganisms. Herein, we examine the effects of exposure to CdtB alone on the small bowel microbiome, and the relationship to the development of IBS-like phenotypes.

Subjects with IBS-D and IBS-C subtypes showed differences in breath gas profile and stool microbiota signature. IBS-D subjects are characterized by an increase in H₂ and H₂S levels with an increase in H₂S producers. IBS-C subjects present an increase in CH₄ and higher presence of M. smithii. Exposure to CdtB alone precipitates altered stool form and profound changes in the small bowel microbiome. These include a subgroup with increased E. coli (a hydrogen producer common in SIBO) and another subgroup with prominent increases in Fusobacterium and Desulfovibrio (a common hydrogen sulfide producer). These findings offer important insights into the potential role of CdtB in the pathophysiology of post-infectious IBS.

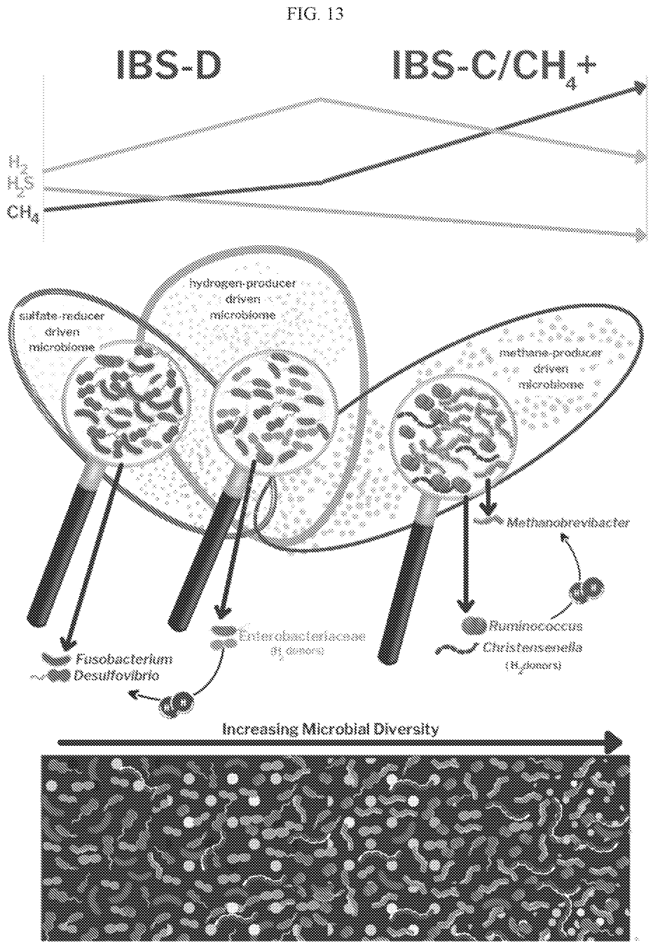

Described herein, we identify breath gas profiles and associated gut microbiome signatures characteristic of IBS phenotypic subtypes, as well as potential microbial drivers (see e.g., FIG. 13). Specifically, in IBS-C subjects with positive CH₄ breath tests, breath CH₄ levels were linked to increased stool levels of the methanogenic archaeon Methanobrevibacter smithii, confirmed by both qPCR and sequencing. These CH₄-positive IBS-C subjects had a distinct gut microbial signature when compared to IBS-D, characterized by increased RA of family Methanobacteriaceae (which includes M. smithii) that correlated with alterations in specific gut bacterial families that include known syntrophs. In contrast, IBS-D subjects were characterized by elevated breath levels of H₂ and H₂S. Breath H₂S levels correlated with RA of gut bacterial H₂S producers, including genus Fusobacterium and an unknown species from genus Desulfovibrio. Induced increases in absolute MJ smithii levels in a rat model resulted in a constipation-like phenotype (decreased stool wet weights), whereas gavage with either of the H₂S-producing species Desulfovibrio piger or Fusobacterium varium resulted in a diarrhea-like phenotype (increased stool wet weights) and stool H₂S production. These findings indicate that increases in these taxa may contribute to the predominant constipation and diarrheal subtypes in IBS subjects.

Although the pathophysiology of IBS has been poorly understood, the gut microbiome appears to play a central role. An early breath testing study suggested the importance of gut microbes in IBS, and three pivotal trials led to FDA approval of an antibiotic treatment for IBS-D. Moreover, a higher proportion of IBS-D patients are H₂-positive compared to controls, and patients with a positive baseline H₂ breath test are more likely to respond to rifaximin. These data suggest that H₂ SIBO is part of the microbiome story in IBS-D. However, H₂ levels do not directly correlate with diarrhea, suggesting that other microbes beyond H₂ producers may also play a role in IBS-D.

CH₄ on breath testing, now categorized as intestinal methanogen overgrowth (IMO) rather than SIBO. CH₄ is produced by methanogenic Archaea, predominantly M. smithii, Methanosphaera stadtmanae and Methanomassiliicoccus luminzyensis, and appears to slow intestinal transit by augmenting segmental smooth muscle contractile activity in the intestinal wall. Like H₂, CH₄ is not produced by human cells.

We found a clear relationship between breath test results and the gut microbiome, with each being a predictor of IBS subtypes. There were significant associations between CH₄ on breath test, gut colonization with M. smithii, and IBS-C. Interestingly, there was also an inverse relationship between CH₄ and H₂ levels. This may be consistent with the syntropic relationship between fermenting bacteria, which produce H₂, and hydrogenotrophic methanogens, which use H₂ to reduce CO₂ into CH₄, preventing accumulation of H₂, which inhibits bacterial growth.

Another interesting finding is that IBS-C/CH₄+ subjects have greater gut microbial diversity than IBS-D. Perhaps reducing localized H₂ concentrations allows specific syntrophic bacterial populations to proliferate, thus increasing diversity. The bacterial genera which co-occur most with methanogens are Christensenella, Bacteroides, Ruminococcus, and Desulfovibrio.

Consistent with this, we found higher RA of families Christensenellaceae and Ruminococcaceae that correlated with RA of Methanobacteriaceae in IBS-C/CH₄+ subjects. Moreover, Ruaud et al. demonstrated that Christensella spp. can transfer H₂ to Methanobrevibacter spp., confirming the syntrophic relationship between these species.

We also identified higher breath H₂S and greater abundance of specific SRB in IBS-D subjects compared to IBS-C/CH₄+. This is consistent with our previous findings linking diarrhea and breath H₂S levels, and independent studies linking H₂S to diarrheal disorders including ulcerative colitis. Supporting this, rat studies have shown that H₂S acts as a smooth muscle relaxant, possibly through direct inhibition of L-type calcium channels. We also found higher RA of genus Fusobacterium, which includes H₂S producers, and an unknown Desulfovibrio species in IBS-D subjects, as well as correlations between Fusobacterium and AUC for H₂S. These data suggest that Fusobacterium and Desulfovibrio spp may drive H₂S production in IBS-D, and thus contribute to the predominant symptom of diarrhea. Further, SRB compete with acetogenic bacteria and archaea for H₂ in the gastrointestinal tract, and we found negative correlations between Fusobacterium spp and M. smithii as well as AUC for CH₄.

Finally, our animal studies further substantiate the direct influence of methanogens and SRB on predominant bowel phenotypes in IBS. Increased M. smithii levels in rats correlated with a constipation-like phenotype based on stool wet weight, whereas increases in F. varium or D. piger (and resulting stool production of H₂S) resulted in a diarrhea-like phenotype. Interestingly, there was a correlation between the KEGG module which predicts the biosynthesis of F420 (an important enzyme in methane production) and breath CH₄ in IBS-C/CH₄+ subjects. Blocking the F420 enzyme with lovastatin lactone or lovastatin hydroxyacid in rats resulted in a reduction in small bowel M. smithii levels and normalization of stool wet weight away from a constipation-like phenotype. Of note, while methanogens are most abundant in the colon in humans, we have previously shown high abundance of methanogens in the small intestine in rats.

In conclusion, our data show that the clinical subtypes of IBS are characterized by unique gut microbial signatures. IBS-C subjects are characterized by increased colonization with methanogenic archaea (predominantly M. smithii), linked to detectable breath CH₄ and a constipation phenotype. In contrast, IBS-D subjects are characterized by increased prevalence of H₂S-producing SRB (predominantly Fusobacterium and Desulfovibrio spp), linked to increased breath H₂S and a diarrhea phenotype. These findings may facilitate a better understanding of the relationship between the gut microbiome and IBS, and the development of microbiome-targeted therapies.

Various embodiments of the present invention are based, at least in part, on these findings.

Various embodiments the present invention provide for a method of distinguishing IBS-D from IBS-C in a subject, comprising: detecting a quantity of Desulfovibrio, a quantity of Fusobacterium, or both in a biological sample from the subject; and identifying the subject as having IBS-D when the quantity of Desulfovibrio, the quantity of Fusobacterium, or both are each higher than its reference quantity. An example of Desulfovibrio includes but is not limited to Desulfovibrio piger. An example of Fusobacterium includes but is not limited to Fusobacterium varium.

Reference quantity can be absolute abundance or a percentage of the total microbiome. For example, the reference quantity of Fusobacterium in stool can be 0.8% of the total microbiome. Additional examples of reference quantity of Fusobacterium, include but are not limited to 1.0%, 0.9%, 0.7%, 0.6%, 0.5%, 0.4%, 0.3% or 0.25%. In another nonlimiting example, the reference quantity for Desulfovibrio can be 0.1% of the total microbiome. Additional examples of reference quantity of Fusobacterium, include but are not limited to 0.09%, 0.08%, 0.07%, 0.06%, 0.05%, 0.04%, 0.03% or 0.025%.

In various embodiments, the method further comprises administering an IBS-D therapy. In various embodiments, the IBS-D therapy comprises rifaximin. In various embodiments, the IBS-D therapy comprises rifaximin and N-acetyl cysteine (NAC).

In various embodiments, the biological sample is whole blood, serum, or plasma. In various embodiments, the biological sample is intestinal aspirate or stool.

In various embodiments, detecting the quantity of Desulfovibrio, the quantity of Fusobacterium, or both comprises using a technique selected from the group consisting of PCR, DNA sequencing to determine the presence of Desulfovibrio DNA or Fusobacterium DNA, culturing for the Desulfovibrio, Fusobacterium or both, 16S rRNA sequencing, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

Various embodiments provide for a method of selecting an IBS therapy for a subject, comprising: detecting a quantity of Desulfovibrio, a quantity of Fusobacterium, or both in a biological sample from the subject; and selecting an IBS-D therapy for the subject when the quantity of Desulfovibrio, the quantity of Fusobacterium, or both are each higher than its reference quantity. An example of Desulfovibrio includes but is not limited to Desulfovibrio piger. An example of Fusobacterium includes but is not limited to Fusobacterium varium.

In various embodiments, the method further comprises administering an IBS-D therapy. In various embodiments, the IBS-D therapy comprises rifaximin. In various embodiments, the IBS-D therapy comprises rifaximin and N-acetyl cysteine (NAC).

In various embodiments, the biological sample is whole blood, serum, or plasma. In various embodiments, the biological sample is intestinal aspirate or stool.

In various embodiments, detecting the quantity of Desulfovibrio, the quantity of Fusobacterium, or both comprises using a technique selected from the group consisting of PCR, DNA sequencing to determine the presence of Desulfovibrio DNA or Fusobacterium DNA, culturing for the Desulfovibrio, Fusobacterium or both, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

Various embodiments of the present invention provide for a method of predicting a subject's response to an IBS-D therapy comprising rifaximin, comprising: detecting a quantity of Desulfovibrio, a quantity of Fusobacterium, or both in a biological sample from the subject; and characterizing the subject as a responder to the IBS-D therapy when the quantity of Desulfovibrio, the quantity of Fusobacterium, or both are each higher than its reference quantity. An example of Desulfovibrio includes but is not limited to Desulfovibrio piger. An example of Fusobacterium includes but is not limited to Fusobacterium varium.

In various embodiments, the method further comprises administering the IBS therapy. In various embodiments, the IBS-D therapy further comprises NAC. As such, the method predicts the subject's response to an IDS-D therapy comprising rifaximin and NAC.

In various embodiments, the biological sample is whole blood, serum, or plasma. In various embodiments, the biological sample is intestinal aspirate or stool.

In various embodiments, detecting the quantity of Desulfovibrio, the quantity of Fusobacterium, or both comprises using a technique selected from the group consisting of PCR, DNA sequencing to determine the presence of Desulfovibrio DNA or Fusobacterium DNA, culturing for the Desulfovibrio, Fusobacterium or both, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

Various embodiments of the present invention provide for a method of predicting a subject's response to an IBS-D therapy comprising rifaximin, comprising: detecting a quantity of hydrogen sulfide (H₂S) in a biological sample from the subject; and characterizing the subject as a responder to the IBS-D therapy when the quantity of H₂S is higher than its reference quantity.

In various embodiments, the IBS-D therapy further comprises NAC. As such, the method predicts the subject's response to an IDS-D therapy comprising rifaximin and NAC.

In various embodiments, the biological sample is a breath sample.

In various embodiments, detecting H₂S in a breath sample is performed by performing a breath test for the presence of H₂S in the subject' breath sample. For example, a lactulose breath test can be administered to the subject, and the breath samples are analyzed for the presence or concentration of H₂S. Examples of reference quantities for H₂S include but are not limited to 3 ppm, 2 ppm, 1.5 ppm, or 1 ppm. In various embodiments, the reference quantity for H₂S is 1.5 ppm.

Various embodiments of the present invention provide for a method of treating IBS-D, comprising: administering an IBS-D therapy comprising rifaximin or an IBS-D therapy comprising rifaximin and NAC to a subject who has been identified as a responder to the IBS-D therapy by a method selected from: (i) detecting a quantity of Desulfovibrio, a quantity of Fusobacterium, or both in a biological sample from the subject, and characterizing the subject as a responder to the IBS therapy when the quantity of Desulfovibrio, the quantity of Fusobacterium, or both are each higher than its reference quantity; or (ii) detecting a quantity of hydrogen sulfide in a biological sample from the subject, and characterizing the subject as a responder to the IBS-D therapy when the quantity of hydrogen sulfide is higher than its reference quantity, or (iii) both.

In various embodiments, the method used to identify the subject comprises detecting a quantity of Desulfovibrio, a quantity of Fusobacterium, or both in a biological sample from the subject, and characterizing the subject as a responder to the IBS therapy when the quantity of Desulfovibrio, the quantity of Fusobacterium, or both are each higher than its reference quantity. An example of Desulfovibrio includes but is not limited to Desulfovibrio piger. An example of Fusobacterium includes but is not limited to Fusobacterium varium.

In various embodiments, the method used to identify the subject comprises detecting a quantity of hydrogen sulfide in a biological sample from the subject, and characterizing the subject as a responder to the IBS-D therapy when the quantity of hydrogen sulfide is higher than its reference quantity.

In various embodiments, the biological sample is a breath sample. In various embodiments, the biological sample is whole blood, serum, or plasma. In various embodiments, the biological sample is intestinal aspirate or stool.

In various embodiments, detecting the quantity of Desulfovibrio, the quantity of Fusobacterium, or both comprises using a technique selected from the group consisting of PCR, qPCT, DNA sequencing to determine the presence of Desulfovibrio DNA or Fusobacterium DNA, culturing for the Desulfovibrio, Fusobacterium or both, and combinations thereof. Examples of DNA sequencing include but are not limited to Sanger sequencing, shotgun sequencing, and high-throughput sequencing (e.g., next-generation “short-read” and third-generation “long-read” sequencing methods (e.g., single molecule real time (SMRT) sequencing, nanopore DNA sequencing).

In various embodiments, the IBS-D therapy decreases hydrogen sulfide produced in the subject's gastrointestinal system. The decrease can be in comparison to the subject's level prior the administration of the IBS-D therapy. The decrease can also be in comparison to a reference value.

The reference value for various embodiments described herein can be established from a biological sample from a healthy individual, or from biological samples from a population of healthy individuals; for example, individuals who do not have IBS, or particularly, do not have IBS-D. For example, if the biological sample is stool, then the reference value can be obtained from the stools of a healthy subject who does not have IBS, or particularly, does not have IBS-D. In some embodiments, the population of healthy subjects can range from at least three healthy individuals to 25 healthy individuals, and even more than 50 healthy individuals. In various embodiments, the population of healthy subjects can range from 25-100 healthy individuals. In various embodiments, the population of healthy subjects can range from 100-250 healthy individuals. In various embodiments, the population of healthy subjects can range from 250-500 healthy individuals. Thus, when a reference value is used, it can be used to determine whether the subject's hydrogen sulfide is lower than the reference value.

In various embodiments, the IBS-D therapy decreases the quantity of Desulfovibrio, a quantity of Fusobacterium, or both in the subject's gastrointestinal system. The decrease can be in comparison to the subject's level of Desulfovibrio, Fusobacterium or both prior the administration of the IBS-D therapy. The decrease can also be in comparison to a reference value. The reference value can be determined from a population of healthy individuals; for example, individuals who do not have IBS. Thus, the reference value can be used to determine whether the subject's Desulfovibrio, Fusobacterium or both is lower than the reference value. The reference value can be established from a biological sample from a healthy individual, or from biological samples from a population of healthy individuals; for example, individuals who do not have IBS, or particularly, does not have IBS-D. For example, if the biological sample is stool, then the reference value can be obtained from the stools of a healthy subject who does not have IBS, or particularly, does not have IBS-D. In some embodiments, the population of healthy subjects can range from at least three healthy individuals to 25 healthy individuals, and even more than 50 healthy individuals. In various embodiments, the population of healthy subjects can range from 25-100 healthy individuals. In various embodiments, the population of healthy subjects can range from 100-250 healthy individuals. In various embodiments, the population of healthy subjects can range from 250-500 healthy individuals. Thus, when a reference value is used, it can be used to determine whether the subject's hydrogen sulfide is lower than the reference value.

In various embodiments, the present invention provides pharmaceutical compositions including a pharmaceutically acceptable excipient along with a therapeutically effective amount of the agent. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylammonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. A thorough discussion is provided in T. Higachi and V. Stella, “Pro-drugs as Novel Delivery Systems,” Vol. 14 of the A. C. S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound (see, e.g., Nogrady (1985) Medicinal Chemistry A Biochemical Approach, Oxford University Press, N. Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Human Clinical Studies Subject Recruitment

Subjects from two randomized controlled trials were included in this study. Baseline breath gases and stool samples from both trials were used. IBS-C subjects were recruited for a clinical trial (clinicaltrials.gov NCT03763175). IBS-C was diagnosed based on Rome IV criteria.

Subjects were excluded if they had recent antibiotics use, had a history of loose or watery stools for >25% of their bowel movements, or had a history of laxative or enema abuse, pelvic floor dysfunction, bariatric surgery, or surgery to remove a segment of the gastrointestinal tract.

The second trial recruited subjects with IBS-D (NCT04557215) based on Rome IV criteria. Subjects were excluded if they had a recent history of antibiotics use, prior known gastrointestinal illness, intestinal surgery, or pelvic floor dysfunction. Both trials were approved by the Cedars-Sinai institutional review board (IRB), and all subjects provided written informed consent.

### Breath Testing

In both studies, subjects underwent baseline (preintervention) lactulose breath testing using a system that allows the measurement of carbon dioxide (CO₂), H₂, CH₄, and H₂S (Gemelli Biotech, Raleigh, NC). Interpretation of breath test results was based on the North American Consensus for breath testing and the ACG Clinical Guideline for SIBO. A positive H₂ breath test was defined as a rise from baseline>20 parts per million (ppm) within 90 minutes. A positive CH₄ breath test was defined as any measurement 10 ppm at any point during the test. As H₂S is not discussed in the consensus or guideline, H₂S levels over the entire breath test were analyzed.

### Stool Sample Collection and Assessment

For both studies, baseline (preintervention) stool samples were self-collected, immediately refrigerated, and then transported to the laboratory. For the IBS-C trial, only CH₄-positive subjects provided stool samples. Upon arrival at the laboratory, an aliquot was transferred to an OMNIgene GUT tube (DNA Genotek, Ottawa, ON, Canada) and stored at room temperature before DNA extraction. Stool form was classified according to the Bristol Stool Scale.

### Stool DNA Extraction

DNA extraction was carried out using the MagAttract PowerSoil DNA KF Kit (Qiagen) with some modifications as described previously. Extracted DNAs were purified using a KingFisher Duo automated system (ThermoFisher Scientific, Waltham, MA), and DNA purity and concentration were determined using a NanoDrop One spectrophotometer (ThermoFisher Scientific).

Determination of Stool Methanogenic Archaea in IBS-C and IBS-D Subjects by qPCR

Levels of two methanogenic archaea, Methanobrevibacter smithii and M. stadtmanae, in stool from IBS-C and IBS-D subjects was determined by quantitative polymerase chain reaction (qPCR) using primers and probes targeting the beta subunit of RNA polymerase (rpoB) gene of each species. Assays were optimized by Applied Biosystems (Custom Taqman Gene Expression Assays). Realtime qPCR was performed on a QuantStudio 6 Flex System (ThermoFisher Scientific) as follows: 1 μL of 20× Custom TaqMan Gene Expression assay solution (ThermoFisher Scientific), 10 μL of TaqMan Fast Advanced Master Mix (ThermoFisher Scientific), 7 μL of PCR grade water and 2 μL of template DNA (25 ng/μl), at 50° C. for 2 min, 95° C. for 2 min, 40 cycles of 95° C. for 1 s, 60° C. for 20 s. DNAs from an M. smithii stock culture and from M. stadtmanae DSM 3091 from the Leibniz Institute DSMZ (Braunschweig, Germany) were extracted using the same protocol, and standard curves with tenfold serial dilutions was prepared for use as qPCR standards.

Library Preparation and 16S rRNA Sequencing

Details of 16S sequencing and analysis protocols are as provided below.

### Animal Studies Animal Models

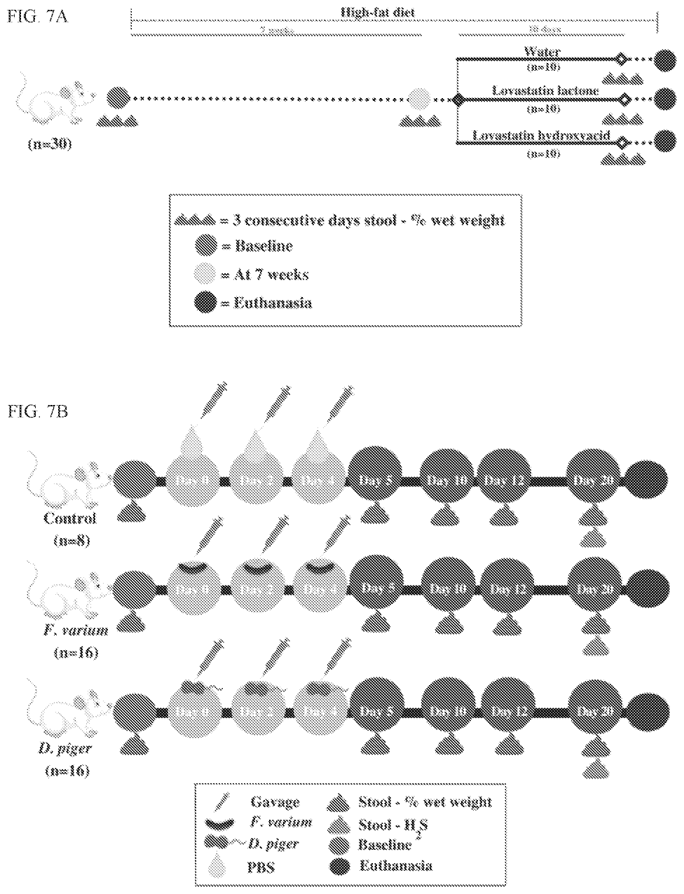

Based on our human studies, there were apparent links between M. smithii and constipation, and between H₂S production and diarrhea. To test whether these represented causal relationships, the effects of M. smithii and two H2S producing bacterial species on stool consistency were determined in vivo (see FIG. 7 for study design). Adult male Sprague Dawley rats (Envigo, Madison, WI) were used for both studies. Both studies were approved by the Cedars-Sinai institutional animal care and use committee (IACUC).

Effects of Methanobrevibacter smithii on Stool Consistency

Previous work showed that Sprague-Dawley rats are endogenously colonized with M. smithii and that M. smithii gavage only transiently augmented its absolute abundance. However, when rats were placed on high-fat diet (HFD), absolute M. smithii levels increased significantly.

Therefore, HFD was used to assess the effects of persistent M. smithii elevation on stool consistency (stool wet weights). Sprague-Dawley rats were placed on a HFD (60% energy from fat [D12492; Research Diets, New Brunswick, NJ]) for 7 weeks. To confirm that decreased stool wet weight was associated with absolute levels of M. smithii, and not a secondary HFD effect, rats were divided into 3 groups and gavaged for 10 days with either 1.5 mg/ml lovastatin lactone, 1.5 mg/ml lovastatin hydroxyacid, or water (as controls) (FIG. 7). Lovastatin is a natural inhibitor of coenzyme F420, a central catabolic cofactor required for methanogenesis. Stool collections were performed at baseline, after 7 weeks on HFD, and after the 10-day lovastatin/control treatments. Stool wet weights were determined. Stool and small bowel DNAs were extracted at euthanasia and the absolute abundance of M. smithii was determined by qPCR as described previously.

### Effects of H₂S Producers on Stool Consistency

The effects of H₂S producers on stool consistency were determined using two sulfate reducing bacteria (SRB), Desulfovibrio piger and Fusobacterium varium. D. piger ATCC29098 (ATCC, Virginia) was grown anaerobically in sterile 1249 modified Baar's medium for sulfate reducers (ATCC) and plated on trypticase soy agar (TSA) with 5% sheep blood (BD, New Jersey). Plates were incubated anaerobically at 37-C for 48-96 hours to obtain single colonies. H₂S production was confirmed.

### SRB Gavage

Rats were divided into three groups and gavaged with: 1) sterile 1×PBS (controls), 2) 1×10⁸ CFU/mL D. piger, or 3) 1×10⁸ CFU/mL F. varium. Liquid cultures were grown from single colonies as described above until 1×10⁸ CFU/mL was achieved. Cultures were centrifuged for 10 min at 3,000×g at 4° C., and bacterial pellets were washed twice and resuspended in 1×PBS to achieve 1×10⁸ CFU/mL. Rats were gavaged on days 0, 2, and 4 (FIG. 7). These rats were fed a standard chow diet (13% energy from fat [PicoLab Rodent Diet 20; LabDiet, St. Louis, MO]).

### Stool Collection and H₂S Measurements

Stool samples were collected by anal stimulation at baseline and on days 5, 7, 10, 12, and 20 (FIG. 7). H₂S production from stool from all rats was measured on day 20. Samples were immediately homogenized with sterile 1×PBS, placed in sterile Erlenmeyer flasks sealed with rubber stoppers connected to a stopcock, and incubated at 37° C. for 2 hours. Gas samples were withdrawn into gas-impermeable bags, and sent for measurement of gases by gas chromatography (Gemelli Biotech). Stool wet weights were measured at all timepoints.

Genomic DNA was isolated from rat stool using a previously-described technique.

### Statistical Analysis

The descriptive analysis is presented as mean: standard deviation. Categorical variables were compared with Chi-square or Fisher's exact tests and continuous variables were compared with t test or Mann-Whitney U-test for two groups. Comparisons between three or more groups were analyzed by one-way ANOVA or Kruskal-Wallis. Correlation between variables were analyzed by Spearman's rank correlation coefficients. Statistical analysis was performed using SPSS 24.0 (SPSS® Inc., Chicago, IL), SAS 9.4 (SAS Institute, Cary, NC), RStudio (RStudio, Boston, MA), and GraphPad Prism® 9 (GraphPad Software, La Jolla, CA). Graph construction was performed using GraphPad Prism 9.02 (GraphPad Software). Significance was set at P<0.05.

Library Preparation and 16S rRNA Sequencing

16S rDNA hypervariable V3 and V4 region libraries were prepared following the Illumina (Illumina, San Diego, CA) protocol (support.illumina.com/documents/documentation/chemistry documentation/16s/16s-metagenomic-library-prep-guide-15044223-b.pdf), using primers S-D-Bact0341-b-S-17 and S-D-Bact-0785-a-A-21. Final libraries were quantified and analyzed on an Agilent 2100 Bioanalyzer System (Agilent Technologies, Santa Clara, CA), again as described previously.

### 16S Amplicon Sequencing and Analysis

Pooled libraries were paired-sequenced (2×301) on an Illumina MiSeq. Reads were quality-based trimmed and merged using the NGS tool available with CLC Genomic Workbench (Qiagen). A reference-based Operational Taxonomic Unit clustering step using the SILVA v132 database was performed with CLC Microbial Genomics Module v.2.5 (Qiagen), using default parameters for minimum occurrences and chimera crossover cost. Creation of new OTUs was not allowed.

Microbial alpha diversity indices including Shannon's, Simpson's, and Observed, were calculated after removal of low depth samples (<5,000 sequences per sample). Inter-sample variability (beta diversity) was calculated using MicrobiomeAnalyst. Differences in microbial relative abundances and microbial alpha and beta diversities between groups were calculated using CLC Microbial Genomics Module v.2.5 (Qiagen) and MicrobiomeAnalyst. Significance was determined by Wald test and Wilcoxon test. Microbial metabolic pathways were analyzed using Kyoto Encyclopedia of genes and Genomes (KEGG) collection databases, using PICRUSt2.

### Confirmation of H₂S Production

H₂S production was confirmed by growing isolated D. piger colonies in 1249 modified Baar's medium for sulfate reducers with 5% of ferrous ammonium sulfate. F. varium clinical isolates were obtained from the Cedars-Sinai Microbiology Department and grown anaerobically in sterile peptone yeast extract broth (Anaerobe Systems, Morgan Hill, CA) and plated on TSA with 5% sheep blood (BD). Isolated single F. varium colonies were cultured in SIM (Sulfide, Indole, Motility) medium (Hardy Diagnostics, CA) to confirm H₂S production.

### Results

### Human Studies Subject Demographics

A total of 171 IBS subjects were included (47 with IBS-D, 124 with IBS-C). Subjects' demographics and clinical characteristics are shown in Table 2. Among IBS-C subjects, 58 (47%) were CH₄-negative (IBS-C/CH₄−), and 66 (53%) were CH₄-positive (IBS-C/CH₄+) and considered to have intestinal methanogen overgrowth (IMO). A total of 8 IBS-D subjects (17%) were CH₄-positive. IBS-C/CH₄+ subjects were significantly older than IBS-D (P=0.037) and IBS-C/CH₄− (P=0.029) subjects. No differences in sex distribution or body mass index (BMI) were identified between groups (Table 2).

TABLE 2
Baseline demographics in IBS-D and IBS-C subjects

| | | IBS-C | | |
|---|---|---|---|---|
| | IBS-D | IBS-C/CH₄− | IBS-C/CH₄+ | P-value^{†} |
| N | 47 | 58 | 66 | |
| Sex (% male) | 36.2 | 29.3 | 24.2 | 0.39 |
| Age (years ± SD) | 45.2 ± | 43.7 ± | 49.6 ± | 0.04 |
| | 15.0 | 14.0 | 10.3*^{‡} | |
| BMI (kg/m² ± SD) | 23.8 ± | 23.4 ± | 24.4 ± | 0.79 |
| | 4.9 | 4.5 | 5.8 | |

Quantitative data are presented as mean ± standard deviation
^{†}P-value based on three group ANOVA
*IBS-C/CR₄+ vs IBS-D P-value < 0.05;
^{‡}IBS-C/CH₄+ vs IBS-C/CH₄− P-value < 0.05

Stool form for all baseline stool samples was assessed using the Bristol stool scale. IBS-D subjects had a significantly higher average score (4.7±1.27), indicating looser, more watery stools, compared to IBS-C/CH4+ subjects (3.2±0.88) (P<0.0001).

Elevated Breath H₂ Levels were More Characteristic of IBS-D

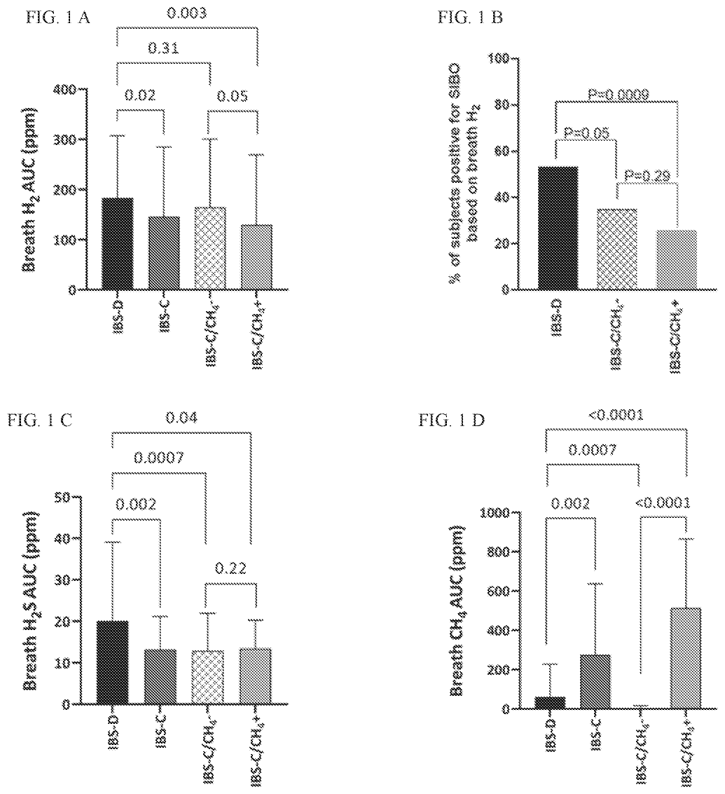

The area-under-the-curve (AUC) for H₂ was higher in IBS-D compared to all IBS-C subjects (P=0.02, FIG. 1A). Moreover, 53.2% of IBS-D subjects were positive for SIBO based on H₂, as were 30.1% of IBS-C subjects (P=0.02). Within IBS-C, 35.09% of IBS-C/CH₄-subjects were positive for H₂ SIBO, compared to 25.76% of IBS-C/CH₄+ subjects (FIG. 1B). Significantly more IBS-D subjects were positive for SIBO compared to IBS-C/CH₄+ (P=0.0009, FIG. 1B).

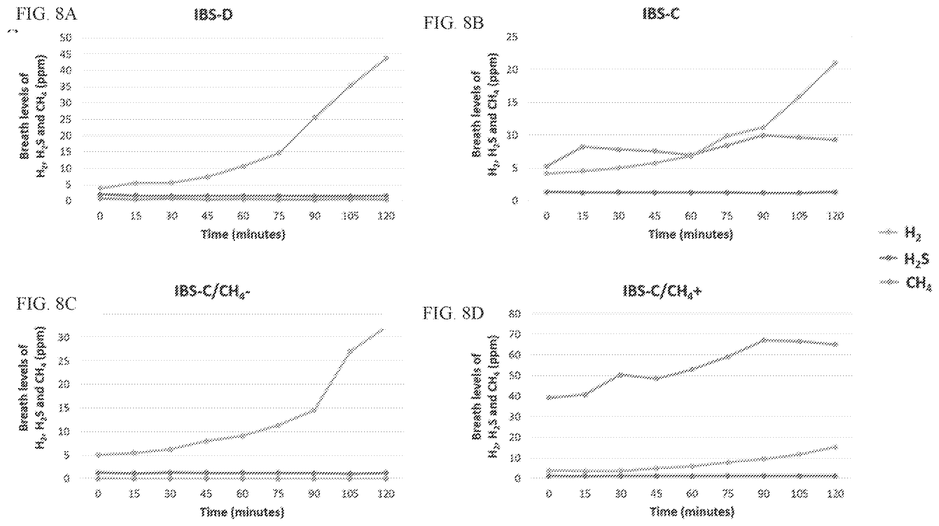

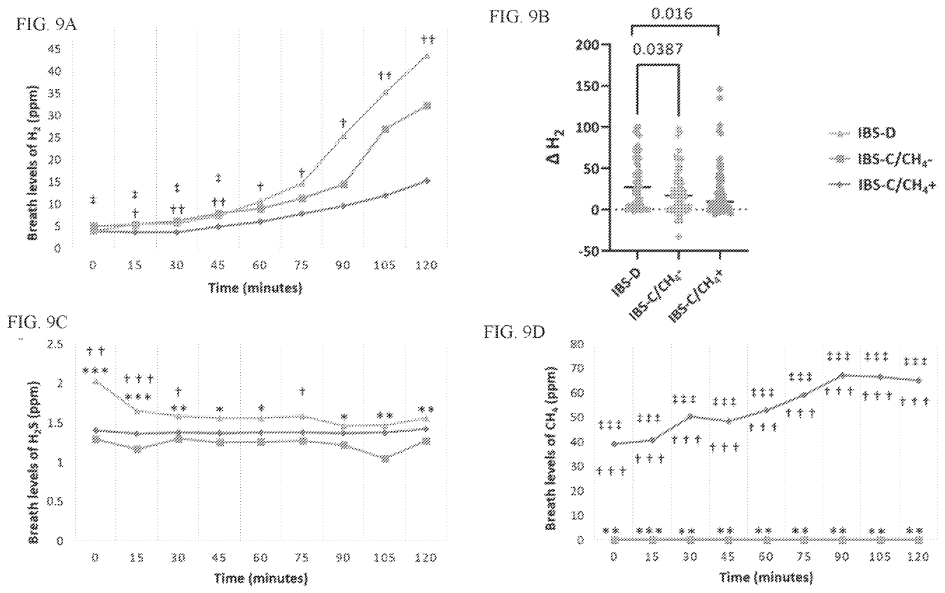

Full breath gas profiles for each group were also explored (FIG. 8, FIG. 9). Breath H₂ dynamics were markedly different between IBS-D subjects and IBS-C subcategories (FIG. 9A). H₂ delta values (120 minutes after lactulose ingestion vs. preingestion levels) were significantly higher in IBS-D subjects vs. IBS-C/CH₄− (P=0.038) and IBS-C/CH₄+ (P=0.016, FIG. 9B). By 15 minutes after lactulose ingestion, H₂ levels were already higher in IBS-D vs. IBS-C/CH₄+ subjects (P=0.037, FIG. 9A), and remained significantly higher at all timepoints during the breath test (Table 1).

TABLE 1
Comparison of H₂, CH₄, and H₂S levels throughout the breath test between
the IBS-D, IBS-C/CH₄− and IBS-C/CH₄+ groups.
HYDROGEN LEVELS

| | | | | Comparison Between Groups (P-values) | | |
|---|---|---|---|---|---|---|
| Breath test | Average Hydrogen Levels (ppm) | | | IBS-D | IBS-D | IBS-C/CH₄− |
| timepoint | IBS-D | IBS-C/CH₄− | IBS-C/CH₄+ | vs | vs | vs |
| (minutes) | (mean ± SD) | (mean ± SD) | (mean ± SD) | IBS-C/CH₄− | IBS-C/CH₄+ | IBS-C/CH₄+ |
| 0 | 5.8 ± 6.1 | 10.0 ± 12.9 | 5.6 ± 8.2 | 0.133 | 0.475 | 0.026 |
| 15 | 7.7 ± 7.8 | 10.3 ± 12.4 | 6.6 ± 9.7 | 0.640 | 0.037 | 0.012 |
| 30 | 8.7 ± 6.9 | 10.8 ± 11.6 | 6.8 ± 9.7 | 0.844 | 0.005 | 0.012 |
| 45 | 11.0 ± 9.9 | 12.4 ± 13.3 | 8.3 ± 10.7 | 0.936 | 0.004 | 0.014 |
| 60 | 16.7 ± 16.2 | 16.2 ± 18.3 | 12.4 ± 15.6 | 0.484 | 0.017 | 0.147 |
| 75 | 22.8 ± 21.4 | 20.2 ± 21.0 | 16.7 ± 21.5 | 0.402 | 0.012 | 0.103 |
| 90 | 30.5 ± 26.3 | 25.6 ± 25.0 | 20.6 ± 26.4 | 0.302 | 0.012 | 0.130 |
| 105 | 37.7 ± 28.8 | 30.9 ± 26.7 | 25.0 ± 30.9 | 0.228 | 0.006 | 0.096 |
| 120 | 43.1 ± 31.9 | 34.5 ± 28.0 | 28.1 ± 34.8 | 0.163 | 0.002 | 0.053 |

METHANE LEVELS

| | | | | Comparison Between Groups (P-values) | | |
|---|---|---|---|---|---|---|
| Breath test | Average Methane Levels (ppm) | | | IBS-D | IBS-D | IBS-C/CH₄− |
| timepoint | IBS-D | IBS-C/CH₄− | IBS-C/CH₄+ | vs | vs | vs |
| (minutes) | (mean ± SD) | (mean ± SD) | (mean ± SD) | IBS-C/CH4 | IBS-C/CH4+ | IBS-C/CH4+ |
| 0 | 6.5 ± 18.5 | 0.5 ± 1.0 | 47.6 ± 31.9 | 0.003 | <0.0001 | <0.0001 |
| 15 | 5.5 ± 16.2 | 0.5 ± 1.2 | 53.8 ± 49.7 | <0.0001 | <0.0001 | <0.0001 |
| 30 | 6.6 ± 18.8 | 0.6 ± 1.5 | 53.4 ± 37.9 | 0.001 | <0.0001 | <0.0001 |
| 45 | 7.1 ± 20.0 | 0.5 ± 1.3 | 54.8 ± 37.5 | 0.001 | <0.0001 | <0.0001 |
| 60 | 6.7 ± 19.6 | 0.6 ± 1.2 | 58.1 ± 40.4 | 0.009 | <0.0001 | <0.0001 |
| 75 | 6.8 ± 20.3 | 0.7 ± 1.5 | 58.6 ± 38.5 | 0.008 | <0.0001 | <0.0001 |
| 90 | 6.8 ± 18.8 | 0.7 ± 1.6 | 63.0 ± 44.4 | 0.006 | <0.0001 | <0.0001 |
| 105 | 7.3 ± 19.2 | 0.7 ± 1.6 | 63.1 ± 45.8 | 0.002 | <0.0001 | <0.0001 |
| 120 | 7.2 ± 20.3 | 0.7 ± 1.6 | 62.9 ± 42.0 | 0.005 | <0.0001 | <0.0001 |

HYDROGEN SULFIDE LEVELS

| | | | | Comparison Between Groups (P-values) | | |
|---|---|---|---|---|---|---|
| Breath test | Average Hydrogen Sulfide Levels (ppm) | | | BBS-D | IBS-D | IBS-C/CH₄− |
| timepoint | IBS-D | IBS-C/CH₄− | IBS-C/CH₄+ | vs | vs | vs |
| (minutes) | (mean ± SD) | (mean ± SD) | (mean ± SD) | IBS-C/CH₄− | IBS-C/CH₄+ | IBS-C/CH₄+ |
| 0 | 2.6 ± 2.3 | 1.4 ± 0.7 | 1.5 ± 0.8 | <0.0001 | 0.002 | 0.411 |
| 15 | 2.4 ± 2.2 | 1.5 ± 1.2 | 1.4 ± 0.7 | <0.0001 | <0.0001 | 0.536 |
| 30 | 2.4 ± 2.2 | 1.5 ± 1.2 | 1.5 ± 0.8 | 0.003 | 0.011 | 0.871 |
| 45 | 2.1 ± 2.2 | 1.5 ± 1.2 | 1.4 ± 0.7 | 0.022 | 0.100 | 0.524 |
| 60 | 2.1 ± 2.2 | 1.4 ± 0.8 | 1.4 ± 0.8 | 0.014 | 0.071 | 0.594 |
| 75 | 2.2 ± 2.2 | 1.4 ± 0.9 | 1.6 ± 1.2 | 0.08 | 0.037 | 0.522 |
| 90 | 2.1 ± 2.1 | 1.6 ± 1.7 | 1.6 ± 1.3 | 0.028 | 0.237 | 0.360 |
| 105 | 2.1 ± 2.2 | 1.5 ± 1.3 | 1.5 ± 0.9 | 0.003 | 0.110 | 0.297 |
| 120 | 2.1 ± 2.0 | 1.5 ± 1.3 | 1.5 ± 0.9 | 0.005 | 0.100 | 0.374 |

Quantitative data are presented as mean ± standard deviation
P-values were determined using Mann Whitney U test

H₂S Levels on Breath Testing were Also More Characteristic of IBS-D

The AUC for H₂S was also higher in IBS-D vs. all IBS-C subjects (P=0.002, FIG. 1C). H₂S levels were also significantly higher in IBS-D vs. IBS-C/CH₄+ subjects at 0 (P=0.002), 15 (P<0.0001), 30 (P=0.011) and 75 (P=0.037) minutes (FIG. 9C, Table 1).

Elevated Breath CH₄ Levels were More Characteristic of IBS-C

The AUC for CH₄ was higher in all IBS-C subjects vs. IBS-D (P=0.002, Table 1, FIG. 1D), driven by the IBS-C/CH₄+ group (P<0.0001). CH₄ dynamics were also different between IBS-C and IBS-D subjects (FIG. 8, FIG. 9), with higher CH₄ levels in IBS-C/CH₄+ vs. IBS-D subjects at all timepoints during the breath test (P<0.0001, FIG. 9D, Table 1).

Associations Between Breath Gases in Subjects with IBS-D and TBS-C/CH₄+

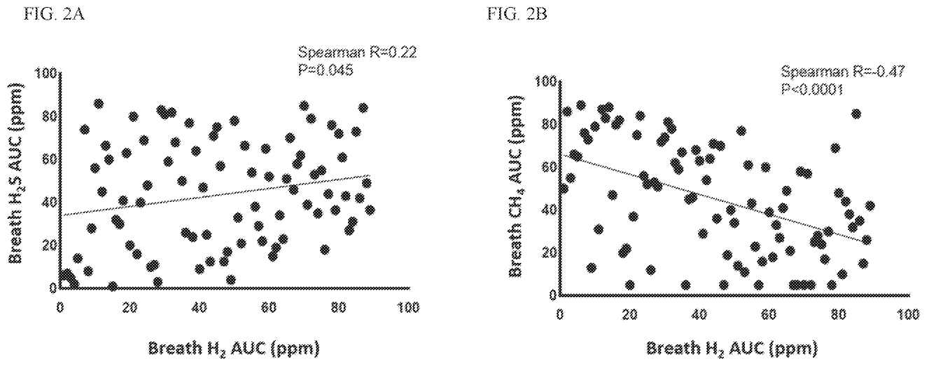

In IBS-D and IBS-C/CH₄+ subjects, breath H₂ AUC correlated positively with breath H₂S AUC (R=0.22, P=0.045, FIG. 2A), but inversely correlated with breath CH₄ AUC (R=−0.47, P<0.0001, FIG. 2B). No association was observed between breath H₂S AUC and CH₄ AUC (P=0.9).

A Gut-Derived Methanogenic Archaeon is Associated with Breath CH₄ Levels

A total of 42 IBS-C/CH₄+ subjects and 40 IBS-D subjects provided baseline stool samples. Of IBS-C/CH₄+ subjects, 88.09% had detectable M. smithii, compared to 17.94% of IBS-D subjects (P<0.0001). M. stadtmanae was less abundant in stool, and was detectable in 10% of IBS-C/CH₄+ and 7.69% of IBS-D subjects (P=1). Absolute M. smithii abundance correlated positively with breath CH₄ levels, regardless of timepoint during the breath test, but a higher correlation coefficient was obtained using the maximum CH₄ level reached during the breath test (R=0.516, P<0.0001). Absolute M. smithii abundance also correlated negatively with breath H₂ levels at 105 minutes (R=−0.375, P=0.008) and 120 minutes (R=−0.332, P=0.02) (Table 3).

TABLE 3
Spearman's correlation between the absolute abundance of
Methanobrevibacter smithii in fecal samples and methane and
hydrogen levels at each timepoint during the lactulose breath test.

| Breath test | Absolute abundance of M. smithii (qPCR) | |
|---|---|---|
| time point | Methane levels | Hydrogen levels |

| (minutes) | R | P-value | R | P-value |
|---|---|---|---|---|
| 0 | 0.410 | 0.003 | −0.047 | 0.750 |
| 1.5 | 0.478 | 0.001 | 0.073 | 0.621 |
| 30 | 0.480 | <0.0001 | 0.045 | 0.759 |
| 45 | 0.533 | <0.0001 | 0.100 | 0.494 |
| 60 | 0.540 | <0.0001 | −0.019 | 0.897 |
| 75 | 0.564 | <0.0001 | −0.179 | 0.217 |
| 90 | 0.545 | <0.0001 | −0.265 | 0.065 |
| 105 | 0.560 | <0.0001 | −0.375 | 0.008 |
| 120 | 0.492 | <0.0001 | −0.332 | 0.020 |

R = Spearman's rank correlation coefficient

### IBS-D and IBS-C are Characterized by Distinct Stool Microbial Signatures

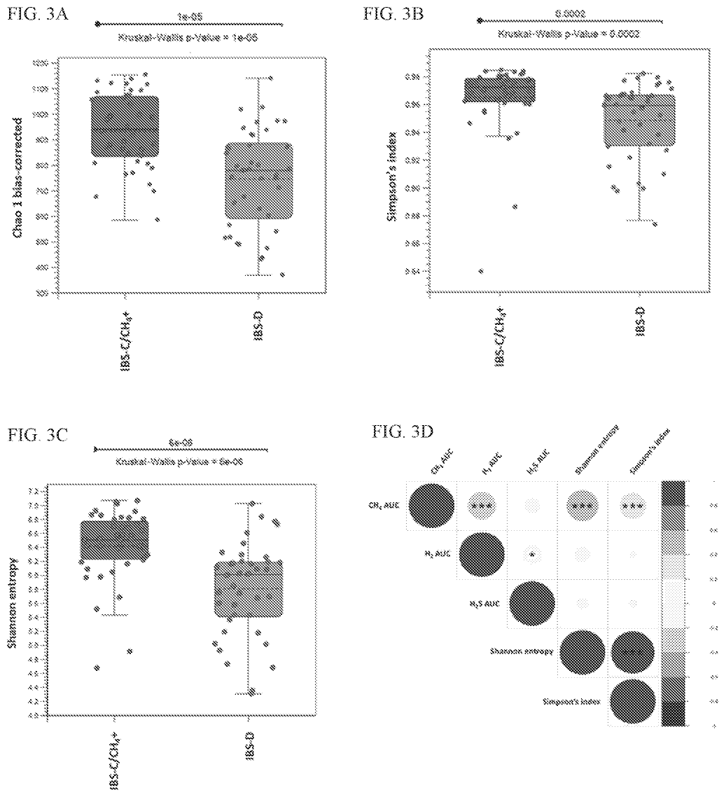

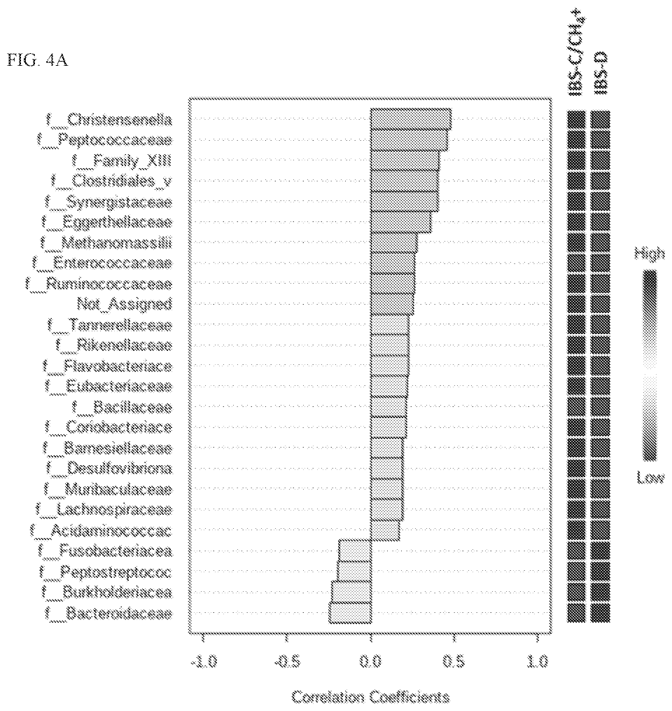

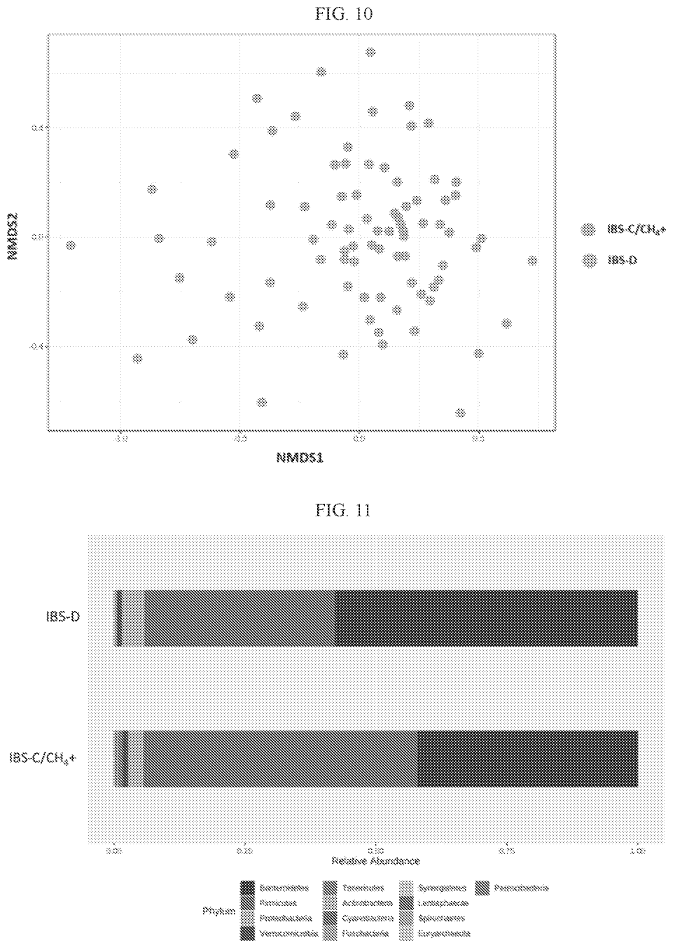

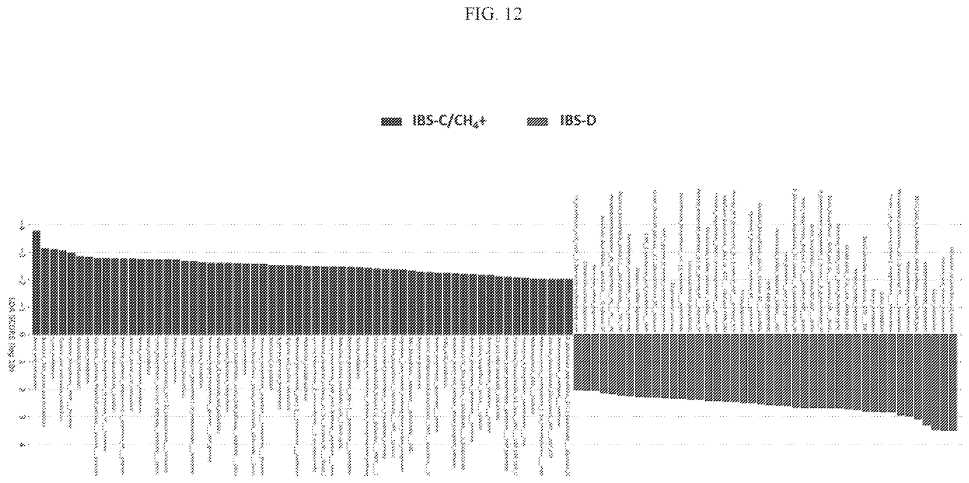

Stool samples (from 42 IBS-C/CH₄+ and 40 IBS-D subjects) were also used for 16S rRNA sequencing. After denoising and removal of low-quality reads, a total of 3,780,543 reads were retained for taxonomic analysis (average 46,104 reads/subject). Microbial alpha diversity analysis revealed a more diverse and enriched stool microbial composition in IBS-C/CH₄+ vs. IBS-D subjects (Chao 1, P=1e-05; Simpson's index P=0.0002 and Shannon's index P=6e-06) (FIG. 3A-C), resulting in distinct microbial signatures on PCA plot [non-metric multidimensional scaling (NMDS), PERMANOVA P<0.001](FIG. 10). Interestingly, stool microbial alpha diversity correlated positively with breath CH4 AUC (Shannon's index R=0.582, P=1.83e-8, Simpson's index R=0.427, P=8.7e-5, FIG. 3D), but correlated negatively with breath H₂ AUC (R=−0.216, P=0.05, FIG. 3D).

Differences in microbial profiles between groups were evident even at higher taxonomic levels. The relative abundance (RA) of microbes from kingdom Archaea, represented by phylum Euryarchaeota, were higher in the stool microbiome of IBS-C/CH4+ subjects compared to IBS-D (Fold change (FC)=8.16, FDR P=2.39E-8). Within kingdom Bacteria, RA of phylum Firmicutes was 1.27-fold higher in IBS-C/CH4+ vs. IBS-D (FDR P=0.04), as were the RA of phyla Tenericutes, Lentisphaerae and Synergistetes were also higher in IBS-C/CH₄+ vs. IBS-D (FC=3.47, FDR P<0.0001; FC=1.74, P=0.006 and FC=2.09, FDR P=0.02 respectively) (FIG. 11).

In contrast, RA of phyla Bacteroidetes (FC=1.39, FDR P=0.02), Fusobacteria (FC=5, FDR P=4.43E-9), Proteobacteria (FC=1.55, FDR P=0.02), Epsilonbacteraeota (FC=2.5, FDR P=3.82E-3) and Spirochaetes (FC=5.91, FDR P=4.43E-9) were higher in IBS-D subjects vs. IBS-C/CH₄+ (FIG. 11).

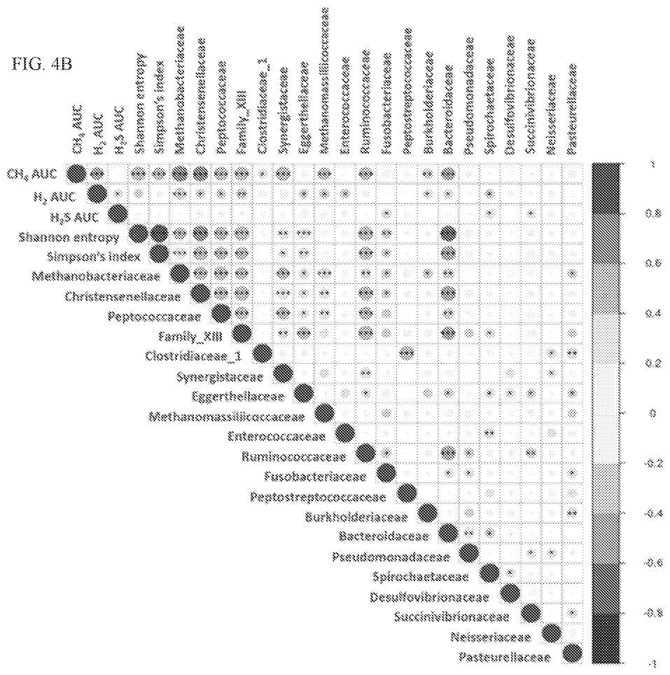

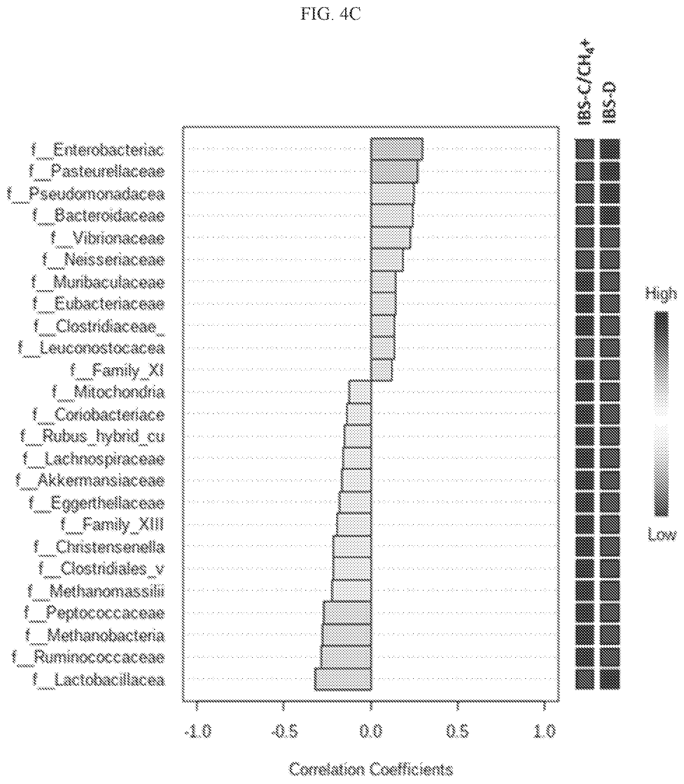

At the family level, the stool microbiome of IBS-C/CH₄+ subjects were characterized by higher RA of methanogenic archaea from Methanobacteriaceae (FC=2.79, FDR P=1.61E-6) and Methanomassiliicoccaceae (FC=2.08, FDR=9.16E-3) when compared to IBS-D subjects. RA of genus Methanobrevibacter was higher in IBS-C/CH4+ subjects vs. IBS-D (FC=2.74, FDR P=1.88E-5), confirming the qPCR results. Bacterial families with higher RA in IBS-C/CH₄+ subjects vs. IBS-D included Anaeroplasmataceae (FC=7.35, FDR P3.11E-12), Flavobacteriaceae (FC=3.84, FDR P=1.48E-5), Christensenellaceae (FC=1.91, FDR P=1.91E-4), Enterococcaceae (FC=2.92, FDR P=2.16E-3), and Ruminococcaceae (FC=1.23, FDR P=0.009), amongst others (Table 4). Notably, RA of family Methanobacteriaceae correlated positively with RA of these bacterial families in IBS-C/CH4+ subjects, indicating possible relationships between these microbes (FIG. 4A). Methanobacteriaceae and the most important associated bacterial families (R>0.25, FIG. 4A) also correlated with breath CH₄ AUC and stool microbial diversity (FIG. 4B).

TABLE 4
Differences in relative abundances of microbial taxa in IBS-D
vs IBS-C/CH₄+ subjects at the phylum, family and genus levels.

| | IBS-D vs IBS-C/CH₄+ | | | |
|---|---|---|---|---|
| | Log₂ fold | Fold | | FDR |
| | change | change | P-value | p-value |
| Phylum | | | | |
| Fusobacteria | 5 | 32.04 | 3.14E−10 | 4.43E−09 |
| Spirochaetes | 5.91 | 60.15 | 5.54E−10 | 4.43E−09 |
| Euryarchaeota | −3.03 | −8.16 | 4.48E−09 | 2.39E−08 |
| Tenericutes | −3.47 | −11.11 | 2.45E−07 | 9.81E−07 |
| Elusimicrobia | −2.9 | −7.47 | 4.91E−04 | 1.57E−03 |
| Epsilonbacteraeota | 2.5 | 5.65 | 1.43E−03 | 3.82E−03 |
| Lentisphaerae | −1.74 | −3.35 | 6.42E−03 | 0.01 |
| Bacteroidetes | 0.47 | 1.39 | 8.48E−03 | 0.02 |
| Synergistetes | −2.09 | −4.25 | 9.21E−03 | 0.02 |
| Proteobacteria | 0.63 | 1.55 | 0.01 | 0.02 |
| Firmicutes | −0.34 | −1.27 | 0.03 | 0.04 |
| Patescibacteria | −0.81 | −1.75 | 0.11 | 0.15 |
| Cyanobacteria | −0.71 | −1.64 | 0.29 | 0.33 |
| Fibrobacteres | −0.82 | −1.76 | 0.29 | 0.33 |
| Verrucomicrobia | −0.46 | −1.37 | 0.51 | 0.54 |
| Actinobacteria | 0.1 | 1.07 | 0.8 | 0.8 |
| Family | | | | |
| Pseudomonadaceae | 7.26 | 153.65 | 0 | 0 |
| Anaeroplasmataceae | −7.35 | −162.8 | 7.88E−14 | 3.11E−12 |
| Bacteroidales | 6.59 | 96.43 | 2.31E−11 | 6.08E−10 |
| Lactobacilliae | 5 | 32.03 | 3.35E−11 | 6.62E−10 |
| Fusobacteriaceae | 5.23 | 37.44 | 2.37E−10 | 3.75E−09 |
| Spirochaetaceae | 5.94 | 61.25 | 4.49E−10 | 5.91E−09 |
| Dysgonomonadaceae | 5.93 | 61.06 | 1.10E−09 | 1.24E−08 |
| Methanobacteriaceae | −2.79 | −6.91 | 1.63E−07 | 1.61E−06 |
| Izimaplasmatales | −3.51 | −11.35 | 1.37E−06 | 1.20E−05 |
| Flavobacteriaceae | −3.84 | −14.36 | 1.87E−06 | 1.48E−05 |
| Clostridiales vadinBB60 group | −2.29 | −4.9 | 5.78E−06 | 4.15E−05 |
| Tectona grandis | 3.06 | 8.32 | 1.43E−05 | 9.44E−05 |
| Christensenellaceae | −1.91 | −3.76 | 3.23E−05 | 1.96E−04 |
| Helicobacteraceae | 3.25 | 9.54 | 2.45E−04 | 1.38E−03 |
| Clostridium sp. K4410.MGS-306 | −2.86 | −7.26 | 3.65E−04 | 1.92E−03 |
| Enterococcaceae | −2.62 | −6.17 | 4.37E−04 | 2.16E−03 |
| Coriobacteriales | −2.16 | −4.46 | 4.80E−04 | 2.23E−03 |
| Elusimicrobiaceae | −2.75 | −6.74 | 8.62E−04 | 3.78E−03 |
| Bacteroidaceae | 0.85 | 1.81 | 1.01E−03 | 4.18E−03 |
| Mollicutes RF39 | −2.3 | −4.92 | 1.16E−03 | 4.60E−03 |
| DTU014 | −1.48 | −2.8 | 1.38E−03 | 5.19E−03 |
| Methanomassiliicoccaceae | −2.08 | −4.22 | 2.55E−03 | 9.16E−03 |
| Victivallaceae | −1.81 | −3.52 | 3.16E−03 | 0.01 |
| Caldicoprobacteraceae | −1.92 | −3.79 | 3.81E−03 | 0.01 |
| Eggerthellaceae | −0.92 | −1.9 | 6.53E−03 | 0.02 |
| Vibrionaceae | 2.01 | 4.03 | 0.01 | 0.03 |
| Defluviitaleaceae | −1.22 | −2.34 | 0.01 | 0.04 |
| Family XIII | −0.93 | −1.91 | 0.02 | 0.05 |
| Burkholderiaceae | 0.97 | 1.95 | 0.03 | 0.07 |
| Leuconostocaceae | 1.67 | 3.18 | 0.03 | 0.08 |
| Peptostreptococcaceae | 0.88 | 1.84 | 0.04 | 0.11 |
| Synergistaceae | −1.6 | −3.03 | 0.04 | 0.11 |
| Bifidobacteriaceae | 1.45 | 2.73 | 0.06 | 0.14 |
| Brachyspiraceae | 1.54 | 2.91 | 0.07 | 0.16 |
| Prevotellaceae | 1.19 | 2.29 | 0.1 | 0.23 |
| Ruminococcaceae | −0.29 | −1.23 | 0.11 | 0.23 |
| Chloroplast | −0.97 | −1.96 | 0.12 | 0.26 |
| Peptococcaceae | −0.59 | −1.51 | 0.18 | 0.37 |
| Family XI-2 | −0.84 | −1.79 | 0.19 | 0.38 |
| Syntrophomonadaceae | −0.99 | −1.98 | 0.21 | 0.4 |
| Rhodospirillales | 0.92 | 1.89 | 0.24 | 0.46 |
| Desulfovibrionaceae | 0.54 | 1.45 | 0.27 | 0.51 |
| Atopobiaceae | −0.77 | −1.71 | 0.28 | 0.52 |
| Coriobacteriales Incertae Sedis | −0.6 | −1.52 | 0.31 | 0.55 |
| Saccharimonadaceae | −0.54 | −1.45 | 0.31 | 0.55 |
| Genus | | | | |
| Pseudomonas | 7.86 | 231.96 | 0.00E+00 | 0.00E+00 |
| Succinivibrionaceae | 7.03 | 130.41 | 2.22E−16 | 3.20E−14 |
| [Eubacterium] oxidoreducens group | −3.43 | −10.82 | 1.65E−14 | 1.59E−12 |
| Fusobacterium | 6.08 | 67.78 | 5.21E−14 | 3.75E−12 |
| [Bacteroides] pectinophilus group | −7.32 | −160.27 | 7.64E−14 | 4.40E−12 |
| Synergistes | −6.8 | −111.26 | 1.89E−12 | 9.08E−11 |
| Anaeroplasma | −6.79 | −110.58 | 2.58E−12 | 1.06E−10 |
| Bacteroidales | 6.73 | 105.9 | 9.35E−12 | 3.37E−10 |
| Treponema 2 | 6.09 | 68.11 | 1.10E−10 | 3.52E−09 |
| Lactobacillus | 4.59 | 24.04 | 3.77E−10 | 1.09E−08 |
| Dysgonomonas | 5.36 | 41 | 1.68E−08 | 4.40E−07 |
| Sarcina | 4.09 | 17.06 | 4.55E−07 | 1.09E−05 |
| Methanobrevibacter | −2.74 | −6.68 | 8.47E−07 | 1.88E−05 |
| Erysipelotrichaceae UCG-006 | 4.35 | 20.46 | 2.15E−06 | 4.42E−05 |
| Coprobacillus | 3.46 | 11.04 | 4.27E−06 | 8.19E−05 |
| Tectona grandis-1 | 3.19 | 9.12 | 4.68E−06 | 8.42E−05 |
| Helicobacter | 4.04 | 16.44 | 7.43E−06 | 1.26E−04 |
| Izimaplasmatales | −3.21 | −9.24 | 8.67E−06 | 1.39E−04 |
| Bacteroides | 1.15 | 2.22 | 9.66E−06 | 1.41E−04 |
| Veillonella | 2.86 | 7.27 | 9.77E−06 | 1.41E−04 |
| GCA-900066225 | −2.55 | −5.88 | 1.07E−05 | 1.46E−04 |
| Flavonifractor | 1.99 | 3.96 | 1.12E−05 | 1.47E−04 |
| Cuneatibacter | −3.11 | −8.62 | 1.86E−05 | 2.33E−04 |
| Ruminococcaceae UCG-011 | −2.59 | −6 | 3.11E−05 | 3.73E−04 |
| CAG-352 | −3.41 | −10.66 | 4.19E−05 | 4.83E−04 |
| Marvinbryantia | −2.04 | −4.12 | 7.80E−05 | 8.64E−04 |
| Flavobacteriaceae | −3.14 | −8.83 | 1.08E−04 | 1.12E−03 |
| [Ruminococcus] gnavus group | 2.54 | 5.8 | 1.07E−04 | 1.12E−03 |
| Anaerosporobacter | 2.83 | 7.13 | 1.28E−04 | 1.23E−03 |
| Oscillospira | −2.31 | −4.94 | 1.26E−04 | 1.23E−03 |
| Phocea | 1.76 | 3.38 | 1.89E−04 | 1.75E−03 |
| Clostridiales vadinBB60 group | −1.9 | −3.73 | 2.12E−04 | 1.91E−03 |
| Christensenellaceae R-7 group | −1.76 | −3.38 | 3.13E−04 | 2.65E−03 |
| Merdibacter | −2.26 | −4.8 | 3.12E−04 | 2.65E−03 |
| Clostridium sp. K4410.MGS-306 | −2.67 | −6.36 | 5.69E−04 | 4.58E−03 |
| Desulfovibrionaceae | 3 | 8.01 | 5.73E−04 | 4.58E−03 |
| Enterobacter | −2.91 | −7.52 | 6.01E−04 | 4.68E−03 |
| Klebsiella | 2.94 | 7.67 | 6.29E−04 | 4.77E−03 |
| Megasphaera | 2.76 | 6.76 | 6.70E−04 | 4.95E−03 |
| Enterococcus | −2.44 | −5.44 | 9.72E−04 | 6.82E−03 |
| UC5-1-2E3 | −2.06 | −4.16 | 9.54E−04 | 6.82E−03 |
| Coriobacteriales | −2.06 | −4.16 | 1.01E−03 | 6.92E−03 |
| Lachnospiraceae | 0.92 | 1.9 | 1.34E−03 | 8.49E−03 |
| Romboutsia | 1.62 | 3.07 | 1.36E−03 | 8.49E−03 |
| [Clostridium] innocuum group | 1.91 | 3.75 | 1.32E−03 | 8.49E−03 |
| Kosakonia | 2.79 | 6.89 | 1.33E−03 | 8.49E−03 |
| Rikenella | −2.65 | −6.27 | 1.40E−03 | 8.58E−03 |
| Mailhella | 2.77 | 6.81 | 1.43E−03 | 8.59E−03 |
| Lachnospiraceae FE2018 group | −2.51 | −5.71 | 1.65E−03 | 9.72E−03 |
| Catenisphaera | −2.55 | −5.86 | 1.92E−03 | 0.01 |
| Anaerovorax | 2.39 | 5.23 | 2.03E−03 | 0.01 |
| Enorma | −2.45 | −5.47 | 2.91E−03 | 0.02 |
| Elusimicrobium | −2.42 | −5.35 | 3.03E−03 | 0.02 |
| Sutterella | 2.15 | 4.43 | 3.03E−03 | 0.02 |
| Eggerthellaceae | −1.79 | −3.47 | 3.27E−03 | 0.02 |
| Lachnospiraceae UCG-004 | 1.42 | 2.67 | 3.20E−03 | 0.02 |
| Moryella | −1.4 | −2.64 | 3.23E−03 | 0.02 |
| Lactococcus | −1.96 | −3.89 | 3.35E−03 | 0.02 |
| Victivallaceae | −2.11 | −4.32 | 4.02E−03 | 0.02 |
| Howardella | 1.9 | 3.73 | 5.09E−03 | 0.02 |
| Mitsuokella | 2.26 | 4.8 | 5.05E−03 | 0.02 |
| Vibrio | 2.18 | 4.53 | 5.59E−03 | 0.03 |
| Ruminococcaceae | −0.82 | −1.77 | 5.90E−03 | 0.03 |
| Acetitomaculum | −1.89 | −3.69 | 6.52E−03 | 0.03 |
| Mollicutes RF39 | −1.97 | −3.91 | 6.75E−03 | 0.03 |
| Rikenellaceae RC9 gut group | 2.4 | 5.27 | 7.01E−03 | 0.03 |
| Ruminiclostridium 1 | −1.62 | −3.08 | 7.11E−03 | 0.03 |
| Veillonellaceae | −2.06 | −4.17 | 7.51E−03 | 0.03 |
| Methanomassiliicoccus | −1.91 | −3.76 | 7.69E−03 | 0.03 |
| Ruminiclostridium | −1.54 | −2.9 | 7.85E−03 | 0.03 |
| Victivallis | −1.64 | −3.12 | 9.14E−03 | 0.04 |
| Ruminococcaceae V9D2013 group | −1.87 | −3.66 | 9.81E−03 | 0.04 |
| Caldicoprobacter | −1.66 | −3.17 | 0.01 | 0.04 |
| Papillibacter | −1.27 | −2.42 | 0.01 | 0.04 |
| Herbinix | 1.61 | 3.04 | 0.01 | 0.04 |
| DTU014 | −1.3 | −2.47 | 0.01 | 0.04 |
| Prevotella 9 | 2.08 | 4.23 | 0.01 | 0.05 |
| Ruminococcaceae UCG-007 | −1.63 | −3.1 | 0.01 | 0.05 |
| Defluviitaleaceae UCG-011 | −1.24 | −2.36 | 0.01 | 0.05 |
| Murimonas | −1.8 | −3.49 | 0.01 | 0.05 |
| Hydrogenoanaerobacterium | −1.42 | −2.67 | 0.01 | 0.05 |
| Prevotella 2 | 2.27 | 4.81 | 0.02 | 0.06 |
| [Eubacterium] hallii group | 0.84 | 1.79 | 0.02 | 0.06 |
| Tyzzerella 3 | 1.78 | 3.44 | 0.02 | 0.07 |
| Gordonibacter | −1.33 | −2.52 | 0.02 | 0.07 |
| Alloprevotella | 2.14 | 4.4 | 0.02 | 0.07 |
| [Eubacterium] fissicatena group | 1.29 | 2.45 | 0.02 | 0.07 |
| [Eubacterium] nodatum group | −1.1 | −2.15 | 0.03 | 0.09 |
| Enterobacteriaceae | 1.73 | 3.32 | 0.03 | 0.1 |
| Citrobacter | 1.46 | 2.74 | 0.03 | 0.1 |
| Proteus | 1.82 | 3.53 | 0.03 | 0.1 |
| Catabacter | −1.32 | −2.5 | 0.03 | 0.11 |
| Bifidobacterium | 1.52 | 2.87 | 0.04 | 0.11 |
| Desulfovibrio | 1.43 | 2.7 | 0.04 | 0.11 |
| Salmonella | 1.77 | 3.41 | 0.04 | 0.11 |
| Lachnospiraceae NK4B4 group | −1.5 | −2.83 | 0.04 | 0.12 |
| Prevotella 6 | −1.57 | −2.97 | 0.04 | 0.12 |
| Streptococcus | 0.93 | 1.91 | 0.04 | 0.12 |
| DTU089 | −0.98 | −1.98 | 0.04 | 0.12 |
| Ruminococcaceae UCG-009 | −0.94 | −1.92 | 0.04 | 0.12 |
| Brachyspira | 1.72 | 3.29 | 0.04 | 0.12 |
| Adlercreutzia | −0.98 | −1.97 | 0.05 | 0.13 |
| Family XIII AD3011 group | −0.99 | −1.99 | 0.05 | 0.13 |
| Faecalicoccus | −1.59 | −3.02 | 0.04 | 0.13 |
| Ruminococcaceae UCG-014 | −1.22 | −2.33 | 0.05 | 0.13 |
| CHKCI001 | 1.36 | 2.57 | 0.05 | 0.13 |
| [Acetivibrio] ethanolgignens group | 1.65 | 3.14 | 0.05 | 0.13 |
| Dubosiella | 1.65 | 3.14 | 0.05 | 0.13 |
| Senegalimassilia | 1.27 | 2.4 | 0.05 | 0.14 |
| Leuconostoc | 1.63 | 3.1 | 0.05 | 0.14 |
| Anaerotruncus | −0.84 | −1.79 | 0.06 | 0.14 |
| Coprococcus 2 | 1.25 | 2.37 | 0.06 | 0.14 |
| Lachnoclostridium 12 | 1.54 | 2.91 | 0.06 | 0.15 |
| Lachnoclostridium | 0.58 | 1.5 | 0.06 | 0.15 |
| Barnesiellaceae | 1.67 | 3.17 | 0.06 | 0.15 |
| CHKCI002 | −1.34 | −2.52 | 0.06 | 0.15 |
| Ruminococcaceae UCG-008 | 1.37 | 2.58 | 0.06 | 0.16 |
| Asteroleplasma | 1.73 | 3.31 | 0.07 | 0.17 |
| Blautia | 0.52 | 1.43 | 0.07 | 0.18 |
| Haemophilus | 1.13 | 2.19 | 0.08 | 0.19 |
| [Eubacterium] coprostanoligenes | −0.73 | −1.65 | 0.09 | 0.2 |
| group | | | | |
| Anaerofilum | −0.98 | −1.97 | 0.09 | 0.2 |
| Harryflintia | −0.77 | −1.71 | 0.09 | 0.2 |
| Prevotella | −1.01 | −2.01 | 0.1 | 0.22 |
| [Eubacterium] ventriosum group | −0.85 | −1.81 | 0.1 | 0.22 |
| [Eubacterium] xylanophilum group | −0.78 | −1.71 | 0.1 | 0.22 |
| CAG-56 | 1.18 | 2.26 | 0.1 | 0.22 |
| Faecalibacterium | 0.62 | 1.53 | 0.1 | 0.23 |
| Weissella | 1.24 | 2.36 | 0.1 | 0.23 |
| Robinsoniella | 1.24 | 2.36 | 0.1 | 0.23 |
| Agathobacter | 0.57 | 1.49 | 0.11 | 0.24 |
| Coprobacter | −1.22 | −2.33 | 0.11 | 0.25 |
| Sanguibacteroides | 1.19 | 2.28 | 0.12 | 0.26 |
| Chloroplast | −0.99 | −1.98 | 0.12 | 0.26 |
| Atopobiaceae | 1.28 | 2.43 | 0.13 | 0.26 |
| Lachnospiraceae UCG-007 | 1.28 | 2.43 | 0.13 | 0.26 |
| Desulfitibacter | 1.29 | 2.45 | 0.12 | 0.26 |
| Comamonas | 1.28 | 2.43 | 0.13 | 0.26 |
| Peptococcus | −0.88 | −1.84 | 0.13 | 0.28 |
| Megamonas | 1.17 | 2.25 | 0.14 | 0.28 |
| Escherichia-Shigella | 0.94 | 1.92 | 0.14 | 0.29 |
| Christensenellaceae | −0.62 | −1.53 | 0.15 | 0.3 |
| Tyzzerella 4 | 1.06 | 2.09 | 0.15 | 0.3 |
| Dorea | 0.6 | 1.51 | 0.15 | 0.3 |
| Peptostreptococcaceae | 0.76 | 1.69 | 0.15 | 0.3 |
| Peptostreptococcus | −0.91 | −1.88 | 0.15 | 0.3 |
| Anaerofustis | −0.76 | −1.69 | 0.15 | 0.3 |
| Lachnospiraceae AC2044 group | −0.86 | −1.81 | 0.15 | 0.3 |
| Oxalobacter | −0.77 | −1.71 | 0.16 | 0.32 |
| Ruminococcus 1 | −0.58 | −1.5 | 0.17 | 0.32 |
| Candidatus Soleaferrea | −0.56 | −1.47 | 0.17 | 0.33 |
| Butyricicoccus | 0.5 | 1.41 | 0.18 | 0.34 |
| Pyramidobacter | −1.05 | −2.07 | 0.18 | 0.34 |
| Anaerovibrio | 1.12 | 2.18 | 0.18 | 0.34 |
| Tectona grandis-2 | 1.12 | 2.18 | 0.18 | 0.34 |
| Prevotellaceae | −1.16 | −2.24 | 0.19 | 0.35 |
| Ruminiclostridium 5 | −0.43 | −1.34 | 0.19 | 0.35 |
| Subdoligranulum | −0.55 | −1.46 | 0.19 | 0.35 |
| Erysipelotrichaceae | −0.93 | −1.9 | 0.19 | 0.35 |
| Holdemania | 0.47 | 1.39 | 0.19 | 0.35 |
| Lachnospiraceae FCS020 group | 0.65 | 1.57 | 0.21 | 0.37 |
| Carya cathayensis | 0.78 | 1.72 | 0.21 | 0.37 |
| Lachnospiraceae UCG-003 | 1.08 | 2.11 | 0.22 | 0.38 |
| Acidaminococcus | −1.08 | −2.11 | 0.22 | 0.39 |
| Prevotella 7 | 1.06 | 2.08 | 0.23 | 0.4 |
| Faecalitalea | −0.75 | −1.69 | 0.23 | 0.4 |
| Solobacterium | 0.88 | 1.84 | 0.23 | 0.4 |
| Phascolarctobacterium | 0.83 | 1.77 | 0.23 | 0.4 |
| Rubus hybrid cultivar-1 | 0.73 | 1.66 | 0.24 | 0.41 |
| Slackia | −0.83 | −1.78 | 0.26 | 0.44 |
| Pseudocitrobacter | −0.88 | −1.84 | 0.26 | 0.44 |
| Lachnoclostridium 5 | −0.69 | −1.61 | 0.26 | 0.44 |
| Tyzzerella | 0.76 | 1.7 | 0.27 | 0.45 |
| Syntrophomonas | −0.85 | −1.8 | 0.27 | 0.45 |
| Muribaculaceae | 0.95 | 1.93 | 0.28 | 0.46 |
| Cloacibacillus | −0.83 | −1.78 | 0.28 | 0.46 |
| Anaerostipes | 0.47 | 1.38 | 0.29 | 0.47 |
| Actinomyces | 0.82 | 1.77 | 0.3 | 0.48 |
| Anaerococcus | 0.82 | 1.77 | 0.3 | 0.48 |
| Pseudoflavonifractor | −0.52 | −1.43 | 0.31 | 0.49 |
| Lachnospiraceae UCG-010 | 0.43 | 1.35 | 0.31 | 0.49 |
| Rothia | 0.54 | 1.45 | 0.31 | 0.49 |
| Ruminococcus 2 | −0.58 | −1.5 | 0.31 | 0.5 |
| Campylobacter | 0.72 | 1.65 | 0.32 | 0.5 |
| Lachnospiraceae UCG-006 | 0.56 | 1.48 | 0.32 | 0.5 |
| Oscillibacter | 0.33 | 1.26 | 0.32 | 0.5 |
| Ruminococcaceae UCG-005 | −0.46 | −1.38 | 0.32 | 0.5 |
| Ruminococcaceae UCG-013 | 0.43 | 1.35 | 0.32 | 0.5 |
| Paraprevotella | 0.81 | 1.75 | 0.33 | 0.5 |
| 28-4 | −0.64 | −1.56 | 0.35 | 0.53 |
| Fournierella | −0.68 | −1.61 | 0.35 | 0.53 |
| Candidatus Stoquefichus | 0.73 | 1.66 | 0.36 | 0.53 |
| Ruminococcaceae UCG-003 | 0.53 | 1.45 | 0.36 | 0.54 |
| Lachnospira | 0.45 | 1.37 | 0.37 | 0.54 |
| Herbaspirillum | −0.7 | −1.63 | 0.37 | 0.54 |
| Marinifilaceae | 0.63 | 1.55 | 0.37 | 0.55 |
| Erysipelatoclostridium | 0.43 | 1.35 | 0.37 | 0.55 |
| Methanosphaera | 0.63 | 1.55 | 0.38 | 0.55 |
| Ezakiella | −0.58 | −1.49 | 0.38 | 0.55 |
| Sellimonas | −0.48 | −1.39 | 0.39 | 0.56 |
| Coriobacteriales Incertae Sedis | −0.51 | −1.42 | 0.39 | 0.56 |
| Lactonifactor | 0.5 | 1.42 | 0.39 | 0.56 |
| Coprococcus 3 | 0.45 | 1.37 | 0.4 | 0.56 |
| Prevotellaceae NK3B31 group | −0.77 | −1.71 | 0.4 | 0.57 |
| Rhodospirillales | 0.63 | 1.54 | 0.41 | 0.58 |
| Atopobium | 0.65 | 1.57 | 0.42 | 0.59 |
| Ruminiclostridium 9 | 0.3 | 1.23 | 0.43 | 0.59 |
| Eisenbergiella | −0.4 | −1.32 | 0.44 | 0.6 |
| Odoribacter | −0.38 | −1.3 | 0.44 | 0.61 |
| Neisseria | −0.58 | −1.49 | 0.44 | 0.61 |
| Negativibacillus | 0.41 | 1.32 | 0.46 | 0.62 |
| Ruminococcaceae UCG-010 | −0.43 | −1.35 | 0.47 | 0.63 |
| Butyricimonas | 0.46 | 1.37 | 0.47 | 0.63 |
| Holdemanella | −0.62 | −1.54 | 0.47 | 0.63 |
| Clostridium sensu stricto 3 | −0.5 | −1.42 | 0.48 | 0.64 |
| [Ruminococcus] torques group | −0.24 | −1.18 | 0.5 | 0.67 |
| Erysipelotrichaceae UCG-003 | 0.39 | 1.31 | 0.5 | 0.67 |
| TM7 phylum sp. oral clone DR034 | −0.36 | −1.28 | 0.51 | 0.67 |
| Peptococcaceae | −0.36 | −1.28 | 0.51 | 0.68 |
| Dielma | 0.32 | 1.24 | 0.52 | 0.68 |
| Shuttleworthia | 0.39 | 1.31 | 0.52 | 0.68 |
| [Ruminococcus] gauvreauii group | 0.25 | 1.19 | 0.53 | 0.69 |
| Parasutterella | 0.37 | 1.3 | 0.53 | 0.69 |
| Ruminiclostridium 6 | −0.4 | −1.32 | 0.54 | 0.7 |
| Granulicatella | 0.3 | 1.23 | 0.55 | 0.7 |
| Lachnospiraceae UCG-001 | 0.3 | 1.23 | 0.55 | 0.7 |
| Catenibacterium | −0.52 | −1.43 | 0.57 | 0.72 |
| Hungatella | −0.29 | −1.23 | 0.59 | 0.74 |
| Fibrobacter | −0.41 | −1.33 | 0.59 | 0.74 |
| Tissierella | −0.41 | −1.33 | 0.59 | 0.74 |
| Pseudobutyrivibrio | 0.4 | 1.32 | 0.6 | 0.75 |
| Allisonella | 0.36 | 1.29 | 0.6 | 0.75 |
| Roseburia | −0.17 | −1.13 | 0.61 | 0.76 |
| Eubacterium | 0.32 | 1.25 | 0.62 | 0.76 |
| Fusicatenibacter | −0.22 | −1.17 | 0.62 | 0.76 |
| vadinBE97 | −0.34 | −1.27 | 0.63 | 0.77 |
| Barnesiella | 3.30E−01 | 1.25 | 0.64 | 0.77 |
| Dialister | 0.34 | 1.27 | 0.64 | 0.77 |
| Bilophila | 2.20E−01 | 1.16 | 0.64 | 0.77 |
| Oribacterium | −3.10E−01 | −1.24 | 0.65 | 0.78 |
| Eggerthella | −0.22 | −1.17 | 0.66 | 0.78 |
| Clostridium sensu stricto 1 | 0.24 | 1.18 | 0.66 | 0.78 |
| Intestinimonas | −0.18 | −1.13 | 0.66 | 0.78 |
| Rubus hybrid cultivar-2 | 0.28 | 1.21 | 0.66 | 0.78 |
| Alistipes | −0.14 | −1.1 | 0.68 | 0.8 |
| UBA1819 | 0.16 | 1.12 | 0.68 | 0.8 |
| Gemella | 0.22 | 1.16 | 0.71 | 0.83 |
| Lachnospiraceae NC2004 group | −0.18 | −1.13 | 0.72 | 0.83 |
| [Eubacterium] eligens group | −0.21 | −1.16 | 0.72 | 0.83 |
| Lachnospiraceae UCG-008 | −0.13 | −1.1 | 0.73 | 0.84 |
| [Eubacterium] brachy group | −0.15 | −1.11 | 0.76 | 0.87 |
| Lachnospiraceae ND3007 group | −0.17 | −1.12 | 0.76 | 0.87 |
| Lachnospiraceae NK4A136 group | 0.13 | 1.09 | 0.76 | 0.87 |
| Erysipelotrichaceae UCG-010 | −0.2 | −1.15 | 0.76 | 0.87 |
| Sneathia | −0.23 | −1.17 | 0.77 | 0.87 |
| Cronobacter | −0.23 | −1.17 | 0.77 | 0.87 |
| Acetanaerobacterium | 0.15 | 1.11 | 0.78 | 0.88 |
| Bacillus | 0.19 | 1.14 | 0.8 | 0.88 |
| Murdochiella | 0.19 | 1.14 | 0.8 | 0.88 |
| Lachnospiraceae XPB1014 group | 0.21 | 1.15 | 0.79 | 0.88 |
| Saccharimonadales | −0.19 | −1.14 | 0.8 | 0.88 |
| Parabacteroides | −0.08 | −1.06 | 0.81 | 0.89 |
| [Eubacterium] ruminantium group | 0.18 | 1.13 | 0.81 | 0.89 |
| Ruminococcaceae NK4A214 group | −0.13 | −1.1 | 0.81 | 0.89 |
| Ruminococcaceae UCG-004 | 0.1 | 1.07 | 0.82 | 0.89 |
| Porphyromonas | 0.13 | 1.09 | 0.84 | 0.9 |
| Butyrivibrio | −0.19 | −1.14 | 0.84 | 0.9 |
| Incertae Sedis | −0.12 | −1.09 | 0.83 | 0.9 |
| Libanicoccus | 0.13 | 1.09 | 0.85 | 0.91 |
| Asaccharobacter | −0.1 | −1.08 | 0.86 | 0.91 |
| Angelakisella | −0.12 | −1.09 | 0.85 | 0.91 |
| Ruminococcaceae UCG-002 | −0.08 | −1.06 | 0.86 | 0.91 |
| Gastranaerophilales | 0.14 | 1.1 | 0.87 | 0.91 |
| GCA-900066755 | 0.11 | 1.08 | 0.87 | 0.91 |
| Olsenella | −0.11 | −1.08 | 0.88 | 0.92 |
| Terrisporobacter | 0.09 | 1.06 | 0.88 | 0.92 |
| Erysipelotrichaceae UCG-004 | 0.08 | 1.06 | 0.9 | 0.94 |
| Akkermansia | 0.08 | 1.06 | 0.9 | 0.94 |
| GCA-900066575 | 0.04 | 1.03 | 0.92 | 0.95 |
| Succinivibrio | 0.1 | 1.07 | 0.92 | 0.95 |
| Turicibacter | −0.05 | −1.03 | 0.93 | 0.96 |
| Collinsella | 0.04 | 1.03 | 0.95 | 0.96 |
| Family XIII UCG-001 | −0.03 | −1.02 | 0.95 | 0.96 |
| Coprococcus 1 | −0.02 | −1.02 | 0.97 | 0.98 |
| Enterorhabdus | −8.03E−03 | −1.01 | 0.99 | 1 |
| Mogibacterium | −0.01 | −1.01 | 0.99 | 1 |
| Paraeggerthella | 7.43E−03 | 1.01 | 0.99 | 1 |
| Gaultheria stenophylla | 2.00E−03 | 1 | 1 | 1 |

The stool microbial signature of IBS-D subjects was characterized by higher RA of several Gram-negative bacteria, including families Pseudomonadaceae (FC=7.26, FDR P<0.00001), Spirochaetaceae (FC=5.94, FDR P=5.91E-9), Fusobacteriaceae (FC=5.23, FDR P=3.75E-9) and Bacteroidaceae (FC=1.81, FDR P=4.18E-3) (Table 4). Most of these microbial families negatively impacted stool microbial diversity and correlated with breath H₂, H₂S, and CH₄ AUC (FIG. 4B).

Fusobacteriaceae (which includes H₂S-producing taxa) and Spirochaetaceae RA correlated with high breath H₂S AUC (R=0.269, P=0.017; R=0.237, P=0.035, respectively, FIG. 4B).

Fusobacteriaceae correlated positively with several families that include Gram-negative bacteria, such as Enterobacteriaceae, Pasteurellaceae, Pseudomonadaceae, Bacteroidaceae and Vibrionaceae (FIG. 4C), but inversely correlated with families associated with IBS-C/CH₄+, including Ruminococcaceae, Methanobacteriaceae, Peptococcaceae and Methanomassiliicoccaceae (FIG. 4C). Details of differences at the genus level are provided in the Supplemental Information.

Gut Microbial Signatures and Predicted Pathways are Associated with Exhaled H₂S and CH₄ in IBS Subjects

Although RA of numerous microbial taxa were different between IBS-C/CH₄+ and IBS-D subjects (Table 4), microorganisms encoding enzymes necessary for H₂S and CH₄ production correlated with breath H₂S and CH₄ levels in IBS subjects. RA of Fusobacterium and an unknown Desulfovibrio species correlated positively with AUC for H₂S (R=0.33, P=0.003; R=0.254, P=0.025, respectively). RA of genus Methanobrevibacter correlated positively with breath CH₄ levels at all timepoints (P<0.0001) and with AUC for CH₄ (R=0.658, P<0.0001), consistent with the findings for stool M. smithii levels by PCR. Moreover, Methanobrevibacter RA correlated negatively with H₂ levels at 105 minutes (R=−0.401, P<0.001) and 120 minutes (R=−0.387, P<0.0001).

Microbial metabolic pathway analysis further supported these associations. A signature associated with biomethanation was characteristic of the stool microbiome in IBS-C/CH₄+ subjects, and included enrichment of KEGG modules associated with methane production from CO₂, methanol, and methylamine (FIG. 12). The KEGG module predicting biosynthesis of F420, a co-factor utilized during methanogenesis, was also enriched in IBS-C/CH₄+ subjects (P<0.0001), and this module correlated with breath CH₄ levels at all timepoints (R=0.488-0.536, P<0.0001), and with CH₄ AUC (R=0.537, P<0.0001).

A biochemical signature associated with sulfur metabolism was characteristic of the stool microbiome in IBS-D subjects, due to enrichment of KEGG modules associated with H₂S production, including dissimilatory and assimilatory sulfate reduction pathways (FIG. 12). Although there were no direct associations between these pathways and breath H₂S levels, the assimilatory sulfate reduction pathway correlated with H₂ levels (R=0.244, P=0.027).

### Animal Studies

Methanobrevibacter smithii Relates to a Constipation-Like Phenotype in Rats

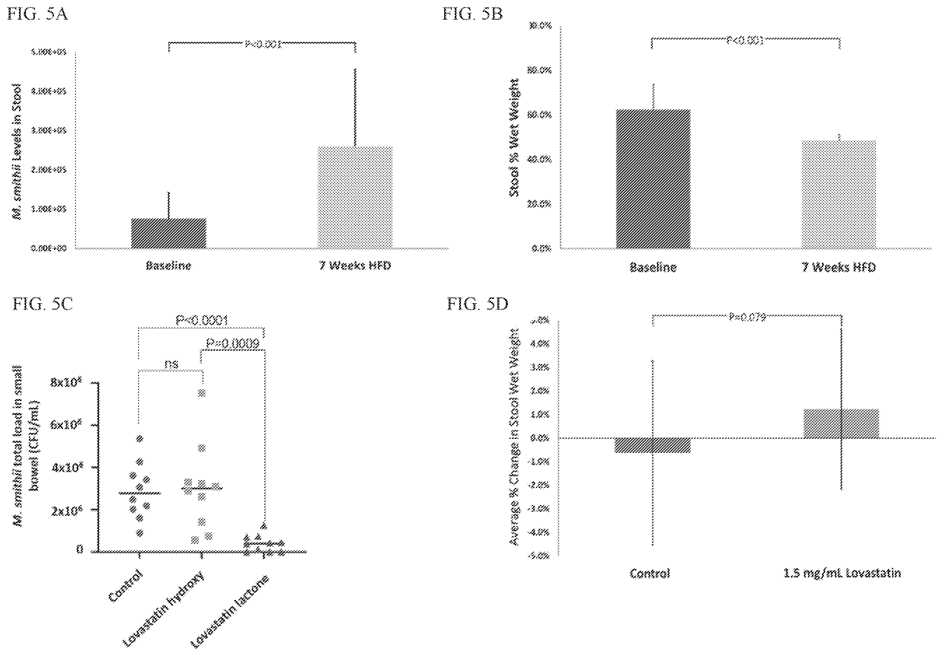

In rats on HFD (N=30), the absolute load of stool M. smithii increased significantly after 7 weeks (2.60×10⁵±1.95×10⁵ CFU/mL) compared to baseline (7.58×10⁴ 6.62×10⁴ CFU/mL, P<0.01, FIG. 5A). Stool wet weight decreased after 7 weeks on HFD compared to baseline (P<0.01, FIG. 5B), and this decrease was associated with the increase in M. smithii absolute load (R=−0.359, P=0.026), indicating that higher M. smithii was associated with drier stool.

Rats treated with the F420 inhibitor lovastatin lactone had decreased M. smithii absolute load in the small bowel compared to controls (P<0.0001, FIG. 5C) and rats treated with lovastatin hydroxyacid (P=0.0009, FIG. 5C). No changes in M. smithii absolute load were observed in the small bowel of rats treated with lovastatin hydroxyacid (FIG. 5C). Although no changes in stool M. smithii loads were observed after treatment, both F420 inhibitors appeared to improve stool consistency of rats on HFD compared to controls on HFD (P=0.07, FIG. 5D).

### Sulfate Reducing Bacteria Induce H₂S Production and a Diarrhea-Like Phenotype in Rats

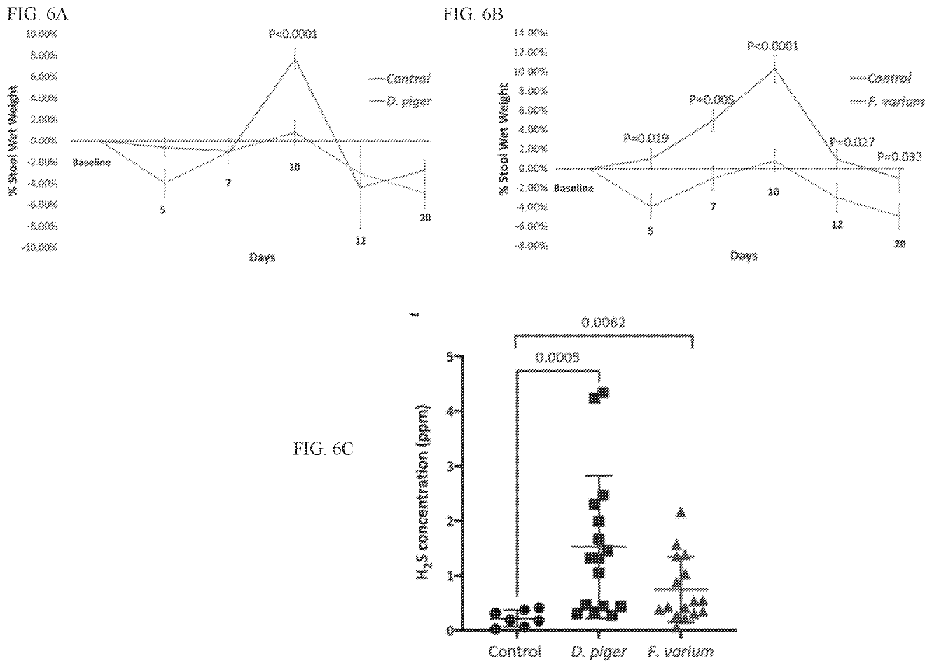

Rats gavaged with D. piger (N=16) had increased stool wet weights on day 10 compared to controls (N=8) (P<0.0001, FIG. 6A). Rats gavaged with F. varium (N=16) had increased stool wet weights on day 5 (P=0.019), day 7 (P=0.005), day 10 (P<0.0001), day 12 (P=0.027) and day 20 (P=0.032) compared to controls (FIG. 6B). Stool H₂S production was greater in rats gavaged with D. piger (N=16, P=0.0005) or F. varium (N=16, P=0.006) vs. controls (N=8, FIG. 6C).

### IBS-D and IBS-C are Characterized by Distinct Stool Microbial Signatures at Genus Level

At the genus level, the relative abundance of RA of 41 known and unknown bacterial genera were higher in the stool microbiome of IBS-C/CH4+ subjects vs. IBS-D (Table 4). The top 5 known genera were Synergistes (FC=6.8, FDR P=9.08E-11), Anaeroplasma (FC=6.79, FDR P=1.06E-10), Cuneatibacter (FC=3.11, FDR P=2.33E-4), Enterobacter (FC=2.91, FDR P=4.68E-3) and Rikenella (FC=2.65, FDR P=8.58E-3).

The RA of 35 known and unknown genera were higher in the stool microbiome of IBS-D subjects vs. IBS-C/CH₄+, including SRB taxa such as genus Fusobacterium (FC=6.08, P=3.75E-12) and an unknown genus from family Desulfovibrionaceae (FC=3, FDR P=4.58E-3) (Table 4). There was no significant difference in RA of genus Desulfovibrio in IBS-D vs. IBS-C/CH4+ subjects after P-value correction (P=0.04, FDR P=0.11), but the RA of several Desulfovibrio OTUs were higher in IBS-D subjects vs. IBS-C/CH₄+ (FDR P<0.05, Table 4).

### Example 2

Methods: 55 male Sprague-Dawley rats were subcutaneously injected with 144 μg recombinant C. jejuni CdtB, and 24 rats were injected with PBS (controls). 3 weeks later, each group received a booster injection of CdtB or PBS, respectively. 12 weeks after the booster, stool wet weights and serum CdtB antibodies (Ab) were measured. Rats were euthanized and ileal luminal contents were collected for V3 and V4 sequencing of microbial DNAs. Operational Taxonomic Unit clustering and taxonomic analysis was performed with CLC Microbial Genomics Module v2.5, and microbial alpha diversity and beta diversity indices were calculated. Significance was determined using the Wald test.

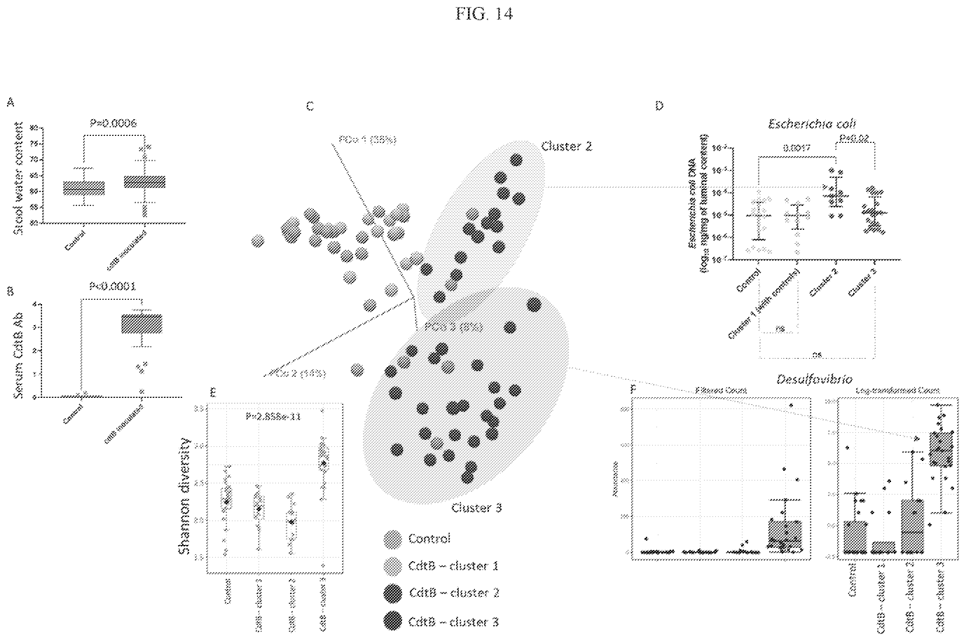

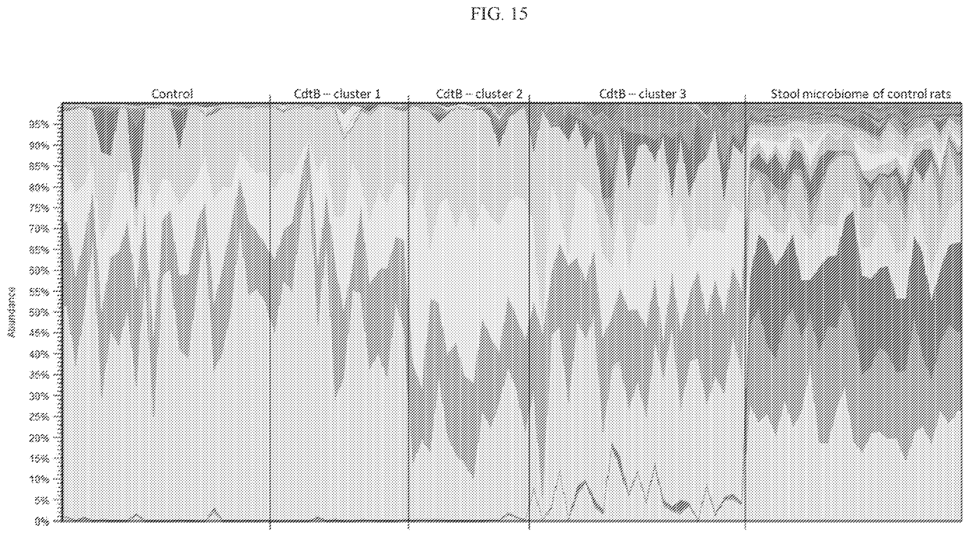

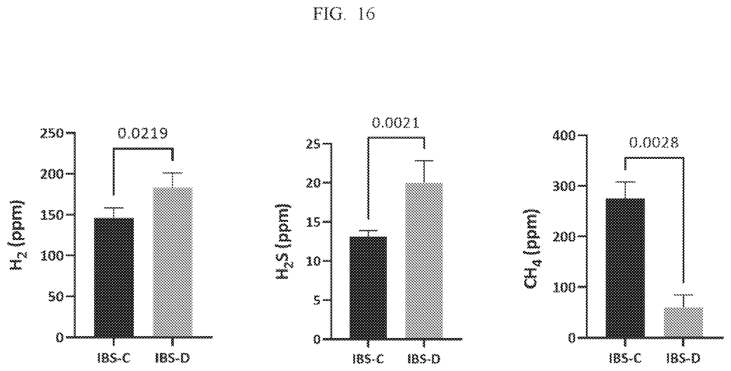

Results: CdtB-inoculated rats had increased stool wet weights (P=0.0006, FIG. 14A), increased CdtB Ab levels (P<0.0001, FIG. 14B), and marked overall changes in the small bowel microbiome (beta-diversity P=6.0e-5, FIG. 14C) when compared to controls. CdtB-inoculated rats exhibited 3 unique microbiome clusters (FIG. 14C). Cluster 1 (n=16) showed no major changes in the ileal microbiome compared to controls (beta-diversity P=0.75, alpha-diversity P=0.202, FIG. 14C, 14E). In fact, lower stool wet weight (PCo1 R=−0.237, P=0.035) and lower CdtB Ab (PCo1 R=−0.366, P=0.001) correlated with cluster 1. Cluster 2 (n=14) had increased absolute abundance of E. coli (P=0.0017, FIG. 14D) (commonly found in SIBO) as well as an increased relative abundance (RA) of other gram-negative bacteria from the family Enterobacteriaceae (Proteus sp. FC=5.74, P=7.8E-5; Morganella sp. FC=5.48, P=3.65E-4), both of which were associated with decreased ileal microbial diversity (R=−0.404, P=0.014, FIG. 14E). Cluster 3 (n=25) driven primarily by increased relative abundances of Desulfovibrio (FC=26.62, P=3.11E-8, FIG. 14F), which strongly correlated with microbial diversity (R=0.728, P<0.0001), and other microbial taxa usually found in stool (a classic signature of fecalization of the small bowel) (FIG. 15).

### Example 3

Methods: IBS subjects from two randomized control trials (RCTs) were recruited and breath levels of H₂, H₂S and CH₄ gases were measured. Analyses were performed using the AUC of each gas. Stool samples were collected using OMNIgene-GUT tubes and DNA was extracted. 16S rDNA hypervariable V3 and V4 regions libraries were prepared and sequenced using MiSeq platform. Detection and quantification of Methanobrevibacter smithii was performed by TaqMan based real-time polymerase chain reaction (RT-qPCR) assay for the rpoB gene. Reference-based Operational Taxonomic Unit clustering was performed using SILVA v132 database. Taxonomic analysis was performed with CLC Microbial Genomics Module v.2.5 and MicrobiomeAnalyst. Significance was determined by Wald test. Microbial metabolic pathways were analyzed with PICRUSt2.

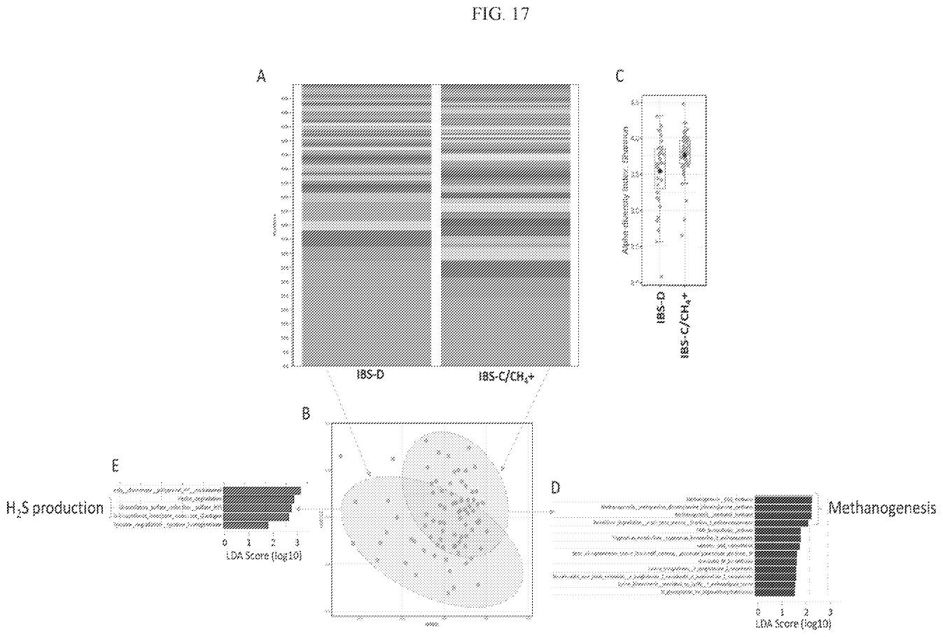

Results: 47 IBS-D and 124 IBS-C were included. Breath H₂ and H₂S were higher in IBS-D compared to IBS-C subjects (P=0.022 and P=0.002, respectively), while CH₄ levels were higher in IBS-C (P=0.003) (FIG. 16). Stools from all IBS-D subjects and 40 CH₄ positive IBS-C subjects (CH₄ levels>10 ppm) were analyzed. The stool microbiome signature of IBS-D and IBS-C were markedly distinct (FIG. 17A), characterized by differences in β-diversity (P<0.001) (FIG. 17B) and α-diversity (Shannon index, P=0.023) (FIG. 17C). The microbial signature observed in IBS-C was associated with increased relative abundance (RA) of microorganisms specialized in methanogenesis (FIG. 17D), including M. smithii—present in 89.36% of IBS-C/CH₄+ subjects, as compared to only 17.5% in the IBS-D (P<0.0001). M. smithii correlated with breath CH₄ (R=0.830, P<0.0001), but a negative association was observed between M. smithii and H₂S producers (R=−0.239, P=0.032). On the other hand, the stool microbial signature in IBS-D was associated with increased RA of H₂S producers (FIG. 17E), including unknown species from Desulfovibrio (P<0.0001) and Fusobacterium (P<0.0001) genera. The RA of H₂S producers was positively associated with breath H₂S (Fusobacterium R=0.33, P=0.003; Desulfovibrio sp. R=0.254, P=0.025).

### Example 4

A patient present with symptoms of irritable bowel syndrome that are inconclusive of whether it may be IBS-C or IBS-D. The patient is tested for Desulfovibrio, Fusobacterium, or both from a small intestinal aspirate sample. The results indicate that the patient has a high quantity of Desulfovibrio compared to its reference quantity. The patient is diagnosed with IBS-D. Further, the patient is administered an IBS-D therapy, such as rifaximin.

### Example 5

A patient present with symptoms of IBS. The patient is tested for Desulfovibrio, Fusobacterium, or both from a duodenal aspirate sample. The results indicate that the patient has a high quantity of Desulfovibrio compared to its reference quantity, and a high quantity of Fusobacterium compared to its reference quantity. The patient is expected to respond to rifaximin treatment. Thus, the patient is administered rifaximin. Optionally, the patient is also administered NAC.

### Example 6

A patient present with symptoms of IBS. The patient is tested the presence of hydrogen sulfide in a breath sample obtained from the patient. The results indicate that the patient has a levels of H₂S its reference quantity. The patient is expected to respond to rifaximin treatment. Thus, the patient is administered rifaximin. Optionally, the patient is also administered NAC.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of distinguishing IBS-D from IBS-C in a subject, comprising:
detecting a quantity of Desulfovibrio, a quantity of Fusobacterium, or both in a biological sample from the subject; and
identifying the subject as having IBS-D when the quantity of Desulfovibrio, the quantity of Fusobacterium, or both are each higher than its reference quantity.

2. The method of claim 1, further comprising administering an IBS-D therapy.

3. The method of claim 2, wherein the IBS-D therapy comprises rifaximin.

4. The method of claim 3, wherein the IBS-D therapy further comprises N-acetyl cysteine (NAC).

5. A method of selecting an IBS therapy for a subject, comprising:
detecting a quantity of Desulfovibrio, a quantity of Fusobacterium, or both in a biological sample from the subject; and
selecting an IBS-D therapy for the subject when the quantity of Desulfovibrio, the quantity of Fusobacterium, or both are each higher than its reference quantity.

6. The method of claim 5, wherein the IBS-D therapy comprises rifaximin.

7. The method of claim 6, wherein the IBS-D therapy further comprises N-acetyl cysteine (NAC).

8. The method of claim 5, wherein the biological sample is a stool sample.

9. The method of claim 5, further comprising administering the IBS-D therapy.

10. A method of predicting a subject's response to an IBS-D therapy comprising rifaximin, comprising:
detecting a quantity of Desulfovibrio, a quantity of Fusobacterium, or both in a biological sample from the subject; and
characterizing the subject as a responder to the IBS-D therapy when the quantity of Desulfovibrio, the quantity of Fusobacterium, or both are each higher than its reference quantity.

11. The method of claim 10, wherein the IBS-D therapy further comprises N-acetyl cysteine (NAC).

12. The method of claim 10, wherein the biological sample is a stool sample.

13. The method of claim 10, further comprising administering the IBS therapy.

14. A method of predicting a subject's response to an IBS-D therapy comprising rifaximin, comprising:
detecting a quantity of hydrogen sulfide (H₂S) in a biological sample from the subject; and
characterizing the subject as a responder to the IBS-D therapy when the quantity of H₂S is higher than its reference quantity.

15. The method of claim 14, wherein the IBS-D therapy further comprises N-acetyl cysteine (NAC).

16. The method of claim 14, wherein the biological sample is a breath sample.

17. The method of claim 14, wherein the biological sample is whole blood, serum, or plasma.

18. A method of treating IBS-D, comprising:
administering an IBS-D therapy comprising rifaximin or an IBS-D therapy comprising rifaximin and N-acetyl cysteine (NAC) to a subject who has been identified as a responder to the IBS-D therapy by a method selected from:
(i) detecting a quantity of Desulfovibrio, a quantity of Fusobacterium, or both in a biological sample from the subject, and characterizing the subject as a responder to the IBS therapy when the quantity of Desulfovibrio, the quantity of Fusobacterium, or both are each higher than its reference quantity; or
(ii) detecting a quantity of hydrogen sulfide in a biological sample from the subject, and characterizing the subject as a responder to the IBS-D therapy when the quantity of hydrogen sulfide is higher than its reference quantity.

19. The method of claim 18, wherein the IBS-D therapy decreases hydrogen sulfide produced in the subject's gastrointestinal system.

20. The method of claim 18, wherein IBS-D therapy decreases the quantity of Desulfovibrio, a quantity of Fusobacterium, or both in the subject's gastrointestinal system.
